(19) United States
(12) Patent Application Publication
(10) Pub. No.: US 2025/0026848 A1
(43) Pub. Date: Jan. 23, 2025

(54) IDENTIFICATION OF PATHOGENIC IMMUNE CELL SUBSETS IN CHECKPOINT INHIBITOR-INDUCED MYOCARDITIS

(71) Applicants: U.S. GOVERNMENT AS REPRESENTED BY THE DEPARTMENT OF VETERANS AFFAIRS, Washington, DC (US); THE BOARD OF TRUSTEES OF THE LELAND STANFORD JUNIOR UNIVERSITY, Stanford, CA (US)

(72) Inventors: Sean M. Wu, Stanford, CA (US); Han Zhu, Stanford, CA (US); Patricia Nguyen, Stanford, CA (US)

(21) Appl. No.: 18/684,342

(22) PCT Filed: Aug. 18, 2022
(86) PCT No.: PCT/US2022/040789

§ 371 Date: Feb. 16, 2024

Related U.S. Application Data
(60) Provisional application No. 63/235,580, filed on Aug. 20, 2021.

(51) Int. Cl. C07K 16/28; A61K 39/00; A61K 45/06; C12Q 1/6883; G01N 33/68

(52) U.S. Cl. CPC C07K 16/289; A61K 45/06; C12Q 1/6883; G01N 33/6863; A61K 2039/505; C12Q 2600/158; G01N 2333/521; G01N 2800/52

(57) ABSTRACT

This disclosure relates to methods helpful in diagnosing and treating immune check point inhibitor (ICI)-induced myocarditis as well as to using C-C chemokine receptor type 1 (CCR1), C-C chemokine receptor type 2 (CCR2), C-C chemokine receptor type 3 (CCR3) and C-C chemokine receptor type 5 (CCR5) antagonists and anti-CD45RA antibodies for treating ICI-induced myocarditis.

### CROSS-REFERENCE TO RELATED APPLICATIONS

This application claims the benefit of priority to U.S. Provisional Patent Application 63/235,580 filed Aug. 20, 2021, the entire disclosure of which is herein incorporated by reference in its entirety.

### STATEMENT OF GOVERNMENT SUPPORT

This invention was made with government support under contract HL149188 awarded by the National Institutes of Health. The government has certain rights in the invention.

### TECHNICAL FIELD

This disclosure relates to compositions and methods for diagnosing and treating immune-related adverse events (IrAE) resulting from a treatment with an immune check point inhibitor (ICI), and in particular ICI-induced myocarditis.

### BACKGROUND

Immune checkpoint inhibitors (ICIs) are life-saving monoclonal antibodies that target intrinsic immune regulatory pathways on T-cells (e.g., cytotoxic T-cell antigen-4/CTLA4 or programmed death-1 protein/PD-1) and release the brakes of T-cell cytotoxicity against tumor cells. Despite their beneficial effects, ICIs can cause immune related adverse events (irAE), autoimmune side effects in various organ systems. One particularly concerning irAE is life-threatening myocarditis, a rare but serious side effect of ICI with up to 50% mortality in affected patients leading to significant long term cardiac side effects including arrhythmias and heart failure (1-3).

Myocarditis is a disease with a heterogenous group of etiologies (viral, drug-induced, idiopathic, etc.) and remains poorly understood despite a high mortality rate (4,5). Due to the often fulminant clinical course of myocarditis, there is significant interest in biomarker discovery for both diagnostic and therapeutic purposes (6). In particular, while mouse immune profiling data exist (6-8), there remains a significant need for comprehensive human immune phenotyping data to find personalized biomarkers and molecular signatures for myocarditis (6,9). In ICI-induced myocarditis, it is critical to quickly and accurately diagnose this disease and determine whether to continue or discontinue ICI therapy and start immunosuppressive treatment in order to minimize cardiac morbidity and mortality (10). Current immunosuppressive treatments, such as glucocorticoids, are largely non-immune cell selective and confer their own nonspecific side effects (11), further highlighting the need to better understand the cellular subsets and mechanisms involved in ICI myocarditis (8).

Histologically, patients with ICI-induced myocarditis have acute lymphocytic infiltrates in the myocardium, implicating T-cell-mediated mechanisms (12-14). Previous mouse studies have reported CD8>CD4 lymphocytic heart infiltration in mice models of PD-1 blockade (14,15), but the mechanisms by which these cells cause pathogenesis in the heart are not well-understood (7).

Thus, there remains the need in the field for biomarkers that associate with an onset and progression of ICI-induced myocarditis, as well as diagnostic and therapeutic methods for treating ICI-induced myocarditis.

### SUMMARY

This disclosure relates to methods helpful in diagnosing and treating ICI-induced myocarditis as well as to using CCR1, CCR3 and CCR5 antagonists and anti-CD45RA antibodies for treating ICI-induced myocarditis.

In one aspect, this disclosure provides a method for reducing an immune-related adverse event (irAE) in an immune checkpoint inhibitor (ICI)-treated human subject, wherein the irAE includes ICI-induced myocarditis, the method comprising administering to the subject a composition comprising one or more of the following:
i. an inhibitor that antagonizes interaction of chemokine (C-C motif) ligand 5 (CCL5) with at least one of the following receptors: C-C chemokine receptor type 1 (CCR1), C-C chemokine receptor type 3 (CCR3) and/or C-C chemokine receptor type 5 (CCR5);
ii. an inhibitor that antagonizes action of the C-C chemokine receptor type 2 (CCR2);
iii. an antibody binding to and downregulating cytotoxic CD8+T effector cells that express one or more cytotoxicity markers and at least the following myocardial-tropic chemokines CCL3, CCL4, CCL4L2 and CCL5 (Temra CD8+ cells); and/or
iv. an antibody binding to and downregulating monocytes/macrophages that express one or more cytotoxicity markers and either C-C chemokine receptor type 2 (CCR2) or C-C chemokine receptor type 5 (CCR5).

Some preferred embodiments of the method may be further characterized by one or more of the following features:
the cytoxicity markers include one or more of the following cytoxicity markers: GZMB, PRF1, KLRK1, KLRB1, KLRF1, and/or IL32;
the Temra CD8⁺ cells express activation marker HLA-DRA;
the macrophages/monocytes expressing either CCR2 or CCR5;
the ICI-myocarditis was diagnosed on basis of elevated cardiac biomarker troponin I over the normal range in the general population, clinical syndrome, negative coronary work-up and/or an imaging diagnosis which included magnetic resonance imaging and/or positron emission tomography (PET); and/or
the reduction in the immune-related adverse event (irAE) is determined by measuring troponin levels prior to administering the composition and recorded as T1, and after administering the composition and recorded as T2, the reduction being achieved if T1 is greater than T2.

The methods include embodiments, wherein the ICI is a monoclonal antibody or an antigen-binding fragment thereof, and wherein the antibody or the fragment selectively binds a regulatory protein present on a T cell, or a ligand for the protein, and thereby activates T-cell cytotoxicity against tumor cells. The methods include embodiments, wherein the regulatory protein is cytotoxic T-cell antigen-4 or programmed death 1 protein (PD-1) or wherein the ligand is programmed death 1 ligand (PDL-1). The methods include embodiments, wherein the ICI is one or more of the following: Ipilimumab (YERVOY), Nivolumab (OPDIVO), Pembrolizumab (KEYTRUDA), Atezolizumab (TECENTRIQ), Durvalumab (IMFINZI), Avelumab (BAVENCIO), and/or Cemiplimab-rwlc (LIBTAYO).

Preferred inhibitors may include one or more of the following:
an inhibitor of CCR1 selected from CP-481,715 (Pfizer), iMLN3897 (Millennium), BX471 (Berlex/Scherring AG) and AZD-4818 (Astra-Zeneca);
an inhibitor of CCR3 selected from SB297006, SB328437, and GW766994;
an inhibitor of CCR5 selected from Maraviroc (monocarboxylic acid amide obtained by formal condensation of the carboxy group of 4,4-difluorocyclohexanecarboxylic acid and the primary amino group of (1S)-3-[(3-exo)-3-(3-isopropyl-5-methyl-4H-1,2,4-triazol-4-yl)-8-azabicyclo[3.2.1]oct-8-yl]-1-phenylpropylamine), leronlimab (PRO 140, a humanized IgG4, kappa monoclonal antibody that recognizes the C-C chemokine receptor type 5, CytoDyn), cenicriviroc (CCR2/5 antagonist), Met-CCL5 (CCL5 antagonist).

Some embodiments of the antibody which is binding to and downregulating the Temra CD8⁺ cells include a monoclonal antibody specific to receptor CD45RA expressed on the Temra CD8⁺ cells, and in particular monoclonal antibody clone HI100 or 5H9 (BD Biosciences), or from clone T6D11 (Miltenyi).

Any of these methods may be further characterized as including those wherein the composition is administered in a therapeutically effective concentration for a period of one to twenty one days, and wherein the therapeutically effective amount is sufficient to reduce the immune-related adverse event (irAE) as determined by measuring troponin levels prior to administering the composition and recorded as T1, and after administering the composition and recorded as T2, the reduction being achieved if T1 is greater than T2. In some embodiments, prior to administering the composition, the subject is tested for levels of CCL5 in the subject's peripheral blood sample and/or for the levels of the Temra CD8⁺ cells in the subject's peripheral blood sample, and in particular the subject may be tested for levels of CCL5 protein in the subject's peripheral blood sample. These methods may further comprise drawing a blood sample from the subject prior to administering the composition.

In some embodiments, the methods may further comprise:
drawing a blood sample from the subject prior to treating the subject with the ICI, after administering the ICI but before administering the composition and after administering the composition, and
measuring the CCL5 protein and/or mRNA levels in the samples;
measuring a number of the Temra CD8⁺ cells in the samples; and/or
measuring numbers of CCR2 or CCR5+ monocytes/macrophages in the samples.

In another aspect, this disclosure relates to methods for testing a human subject undergoing treatment with an immune checkpoint inhibitor (ICI), the method comprising:
1) drawing a first blood sample from the human subject prior to administering the ICI;
2) drawing a second blood sample from the human subject after administering the ICI;
3) measuring CCL5 protein and/or mRNA levels in the first sample and in the second sample; and/or measuring a number of specialized effector CD8⁺ T cells expressing one or more cytotoxicity markers and at least the following myocardial-tropic chemokines CCL4, CCL4L2 and CCL5 (Temra CD8⁺ cells) in the first sample and in the second sample; and/or
4) measuring CCR2/CCR5 protein and/or mRNA levels in the first sample and in the second sample; and/or measuring a number of monocytes/macrophages expressing one or more cytotoxicity markers and at least the following chemokine receptors (CCR2 or CCR5) in the first sample and in the second sample.

Preferably, wherein the level of CCL5 protein and/or mRNA in the first sample is L1, and wherein the level of CCL5 protein and/or mRNA in the second sample is L2, and wherein when L2 is greater than L1, the human subject is eligible for anti-irAE treatment.

Some embodiments of these methods may be further characterized by one or more of the following features:
the CCL5 protein, CCR2 protein, and/or CCR5 protein is detected by ELISA and/or CCL5 mRNA is detected by quantitative PCR; and/or
mononuclear cells are isolated from the blood samples, CD8⁺ T cells or monocytes/macrophages are isolated from the blood samples, and the CCL5, CCR2 and/or CCR5 protein; and/or CCL5, CCR2 and/or CCR5 mRNA is detected in the isolated population of CD8⁺ T cells and/or monocytes and/or macrophages.

Preferably, the number of Temra CD8⁺ cells and/or monocytes/macrophages in the first sample is T1, and wherein the number of Temra CD8⁺ cells and/or monocytes/macrophages in the second sample is T2, and wherein when T2 is greater than T1, the subject is eligible for anti-irAE treatment.

In yet another aspect, this disclosure relates to a use of an inhibitor of interaction of chemokine (C-C motif) ligand 5 (CCL5) with one or more of the following receptors: C-C chemokine receptor type 1 (CCR1), C-C chemokine receptor type 3 (CCR3) and/or C-C chemokine receptor type 5 (CCR5) for treating an immune checkpoint inhibitor (ICI) induced myocarditis; or a use of an antibody binding to and downregulating specialized effector CD8⁺ T cells (Temra CD8⁺ cells) for treating an immune checkpoint inhibitor (ICI) induced myocarditis. Preferably, the inhibitor may be selected from CP-481,715 (Pfizer), iMLN3897 (Millennium), BX471 (Berlex/Scherring AG), AZD-4818 (Astra-Zeneca), SB297006, SB328437, GW766994, Maraviroc (monocarboxylic acid amide obtained by formal condensation of the carboxy group of 4,4-difluorocyclohexanecarboxylic acid and the primary amino group of (1S)-3-[(3-exo)-3-(3-isopropyl-5-methyl-4H-1,2,4-triazol-4-yl)-8-azabicyclo[3.2.1]oct-8-yl]-1-phenylpropylamine), leronlimab (PRO 140), cenicriviroc, and Met-CCL5.

In yet another aspect, this disclosure relates to a use of anti-CD45RA monoclonal antibody for treating an immune checkpoint inhibitor (ICI) induced myocarditis. Preferably, the antibody may be selected from anti-CD45RA monoclonal antibody clone HI100 or 5H9 (BD Biosciences), or from clone T6D11 (Miltenyi).

In yet another aspect, this disclosure relates to a method of identifying a human subject eligible for ICI-myocarditis treatment, wherein the human subject is undergoing treatment with an immune checkpoint inhibitor (ICI), the method comprising analyzing CD8+ T cell population in a peripheral blood sample of the human subject for presence of Temra CD8+ cells expressing myocardial-tropic chemokines: CCL3, CCL4, CCL4L2 and CCL5 and also expressing CD45RA, and wherein when the Temra CD8+ cells are present in the blood sample, the human subject is eligible for ICI-myocarditis treatment. Preferably, the CD8+ T cell population may be analyzed during a time period from day 15 to day 35 after initiation of the ICI therapy. In some embodiments, the analysis may include CyTOF (time of flight mass cytometry) and/or simultaneous single cell RNA-Seq and single cell TCR sequencing. In some embodiments, the method may comprise collecting a blood sample before the ICI treatment begins, said sample being used as a control sample and wherein the human subject is eligible for ICI-myocarditis treatment when the presence of the Tempra CD8⁺ cells is greater in the sample obtained during the ICI-treatment than in the human subject's control sample.

In yet another embodiment, this disclosure relates to a method of treating ICI-induced myocarditis in a human subject, wherein the method comprises monitoring the human subject for a phenotypic shift in the activated Tempra CD8⁺ population from an early cytotoxic and pro-inflammatory profile to a late exhaustion phenotype expressing known markers of T-cell exhaustion, including KLRG, CX3CR1 and/or LAG3.

### BRIEF DESCRIPTION OF THE DRAWINGS

FIG. 1. Analysis of Immune Cell Populations in ICI Myocarditis using CyTOF Reveals Cytotoxic Temra CD8+ Expansion. (A) Workflow showing collection of peripheral blood and processing of single-cell suspensions for mass cytometry (CyTOF). (B) Identification of peripheral blood CD45+ immune cell clusters across all samples (n=4-23 subjects per cohort). (C) Feature plots with canonical markers across CD45+ clusters. (D) Quantification of immune cell distribution across clusters and comparison of patient groups showing a relative increase in monocytes and reduction in T-cells in circulating blood in the ICI-treated patients (Groups A-C) compared to “No ICI” control group, with the myocarditis patients (Group C) showing the largest increase in circulating monocytes. For each cluster, the average fraction of cells from each patient group is shown, after normalization for total CD45+ input cell numbers per patient. Average and SEM are shown for each patient group. Statistical analysis compares groups No ICI, A, B, and C. (E) Identification of peripheral blood CD3+ immune cell clusters across all samples (n=4-23 subjects per group, 40 patients total). (F) Feature plots with canonical markers across CD3+ clusters. (G) Quantification of CD3+ cell subtypes across clusters and comparison of patient groups showing a relative increase in CD8+ compared to CD4+ T-cells in the peripheral blood in the myocarditis group compared to “No ICI” controls but not significantly increased compared to other ICI-treated patients. For each cluster, the average fraction of cells from each patient group is shown, after normalization for total CD3+ input cell numbers per patient. Average and SEM are shown for each patient group. Statistical analysis compares groups No ICI, A, B, and C. (H) Identification of peripheral blood CD4+ immune cell clusters across all samples (n=4-23 subjects per cohort). (I) Identification of peripheral blood CD8+ immune cell clusters across all samples (n=4-23 subjects per cohort). (J) Quantification of CD4+ cell subtypes across clusters and comparison of patient groups. For each cluster, the average fraction of cells from each patient group is shown, after normalization for total CD3+ input cell numbers per patient. Statistics are calculated as described above. (K) Quantification of CD8+ cell subtypes across clusters and comparison of patient groups showing a significant increase in the proportion of Temra CD8+ T-cells in the myocarditis group compared to control groups. For each cluster, the average fraction of cells from each patient group is shown, after normalization for total CD3+ input cell numbers per patient. Statistics are calculated as described above.

FIG. 2. Analysis of Immune Cell Populations in ICI Myocarditis using scRNAseq. (A) Workflow showing collection of peripheral blood and processing of single-cell suspensions for single-cell RNA-seq. (B) Identification of peripheral blood CD45+ immune cell clusters across all samples (n=3-6 subjects per cohort). (C) Feature plots with canonical markers across CD45+ clusters. (D) Feature barcoding with CITE-seq showing surface markers CD45RA, CD4 and CD8, (E) Heatmap of top differentially expressed genes across clusters. (F) Quantification of CD45+ cell subtypes across clusters and comparison of patient groups showing an expansion of monocytes and a relative reduction in circulating T-cells in the ICI-treated groups compared to the “No ICI” control patients. As seen in the CyTOF data, the myocarditis patients (Group C) had increased expansion of circulating monocytes compared to other groups. For each cluster, the average fraction of cells from each patient group is shown, after normalization for total CD3+ input cell numbers per patient. Average and SEM are shown for each patient group. Statistical analysis compares groups No ICI, A, B, and C.

FIG. 3. Myocarditis-Related Temra CD8+ Expansion Confirmed by scRNAseq. (A) Identification of peripheral blood CD8+ immune cell clusters across all samples (n=3-6 subjects per cohort). (B) RNA feature plots with canonical markers across CD8+ clusters. (C) Feature plots of CD45RA surface protein expression using CITE-seq feature barcoding technology. (D) Heatmap of top differentially expressed genes across clusters. (E) Quantification of CD8+ cell subtypes across clusters and comparison of patient groups show a trend towards increased proportion of Temra CD8+ T-cells in the myocarditis group compared to controls. For each cluster, the average fraction of cells from each patient group is shown, after normalization for total CD8+ input cell numbers per patient. Average and SEM are shown for each patient group. Statistical analysis compares groups No ICI, A, B, and C. (F) Feature plots displaying expression of cytotoxicity markers (GZMB, PRF1) as well as the activation marker HLA-DRA in the Temra CD8 clusters. (G) Violin plots showing increased log normalized expression of cytotoxicity genes (GZMB, PRF1) and the activation gene, HLA-DRA, in Temra CD8+ cells compared to the other CD8+ cell types.

FIG. 4. Single-Cell TCR Sequencing Reveals Myocarditis-Associated Clonal Expansion of Temra CD8+ Cell Clusters. (A) Visualization of top 50 TCR clonotypes across patient groups A, B, and C showing expansion of clonotypes in group C (myocarditis) patients compared to control groups. (B) Quantification of top 50 TCR clonotypes across clusters as a fraction of total CD3+ cells across patient groups. For each patient in a group, the fraction of top 50 clonotypes was normalized against CD3+ input cell numbers. Average and SEM are shown for each patient group. Statistical analysis compares groups A, B, and C. (C) Quantification of top 50 TCR clonotypes across CD8+ cell subtypes as previously defined in FIG. 3, compared across patient groups A, B, and C showing increase in Temra CD8+ in the myocarditis group C. (D) Quantification of top 50 TCR clonotypes across the eight CD8+ cell clusters compared across patient groups A, B, and C. Temra CD8+ clusters 0 and 8 show expansion in the myocarditis group C and are highlighted in red. Temra CD8+ clusters 3 and 4 show expansion in non-myocarditis irAE group B and are highlighted in blue. (E) Violin plots displaying significantly differentially expressed genes across CD8+ T cell clusters comparing log normalized expression of significant genes in myocarditis-associated clusters (0,8) highlighted in red vs group B (non-myocarditis irAE) clusters (3,4) highlighted in blue. Clusters 0, 3, 4 and 8, particularly myocarditis-associated cluster 8, show increased activation (HLA-DRA) and cytotoxicity (GZMB, KLRB1, KLRF1, IL32) gene expression. Non-myocarditis irAE-associated cluster 3 demonstrates increased expression of T-cell exhaustion markers CX3CR1, KLRG1, and to a lesser extent, TIGIT. Myocarditis cluster 8 also exhibits increased expression of myocardial-tropic chemokines CCL3/CCL4L2/CCL4/CCL5.

FIG. 5. Activated and Expanded Cytotoxic Temra CD8+ Clones Persist Two Months after Myocarditis but Exhibit Markers of T-Cell Exhaustion. (A) Peripheral blood was collected from a patient with myocarditis (MCE1) at initial myocarditis diagnosis (early timepoint=day 0) and after resolution of clinical myocarditis and troponin I biomarker (late timepoint=day 65). (B) Cardiac magnetic resonance imaging (cMRI) shows myocarditis in the lateral wall at early timepoint (day 0) including myocardial thinning and delayed gadolinium enhancement (DGE) and increased T2 signal consistent with myocardial edema, with resolution myocardial edema by late timepoint (day 65). (C) Clonal analysis of CD3+ cells in peripheral blood by single-cell TCR sequencing shows oligoclonal expansion of top 50 TCR clonotypes (53.9% of all CD3+ cells in peripheral blood) in the early myocarditis timepoint which decreases slightly but persists (36.5% of CD3+ cells) in the late timepoint. Top 10 expanded clonotypes are shown by their individual colors, with top expanded clonotype 1 shown in purple, etc. (D) Visualization of top 10 expanded TCR clonotypes on UMAP showing localization of individual expanded clonotypes in clusters 0 and 8 which translocate to cluster 3 in the late timepoint. (E) Quantification of percentages of top 10 expanded TCR clonotypes in each cluster as a fraction of total CD3+ cells in the patient in the early and late timepoints, showing shift in clonally expanded population from clusters 0 and 8 to cluster 3 in the late timepoint. (F) Sankey diagram showing shift in the top 10 expanded TCR clonotypes from predominantly clusters 0 and 8 to cluster 3 in the late timepoint. (G) Violin plots showing differential gene expression analysis of the top 10 expanded TCR clonotypes across clusters, showing decreased expression of cytotoxicity genes (IL32, KLRK1, IL32) and homing chemokine genes (CCL5, CCL4, CC14L2) in cluster 3 (associated with the late timepoint). In contrast, cluster 3 exhibited higher expression of T-cell exhaustion genes CX3CR1, KLRG1, and LAG3. (H) Violin plots of differential gene analysis in the early vs. late timepoints, showing relative decrease in T-cell cytotoxicity genes (GZMB, KLRK1, IL32, KLRB1, IL2RG) and homing chemokines (CCL5, CCL4, CCL4L2) in the late compared to early timepoints. In contrast, there was relative increased expression of T-cell exhaustion markers (CX3CR1, KLRG1, LAG3, and KLF2) in the late compared to early timepoint. (I) Violin plots showing differential gene expressions in top 10 expanded individual TCR clonotypes in the early vs. late timepoint, demonstrating similar decrease in cytotoxicity/homing genes and increase in exhaustion genes in the late timepoint.

FIG. 6. Gene Expression Programs of Myocarditis-Associated T Cell Populations Exhibit Increased Cardiotropic Chemokines and Markers of Autoimmunity. (A) Feature plots showing top differentially expressed genes in CD8+ T-cells across patient groups A, B, and C, showing increased expression of myocardial-tropic chemokines (CCL4L2, CCL4 and CCL5) and inflammatory interleukin-32 (IL32). (B) Violin plots showing averaged log-fold expression of top differentially expressed genes across the patient groups A, B and C. (C) Predicted ligand-receptor interactions of Temra CD8+ cells in entire patient cohort. Interaction scores were calculated based on the expression of ligands and corresponding receptors in scRNA-seq data [CellPhoneDB, (https://www.cellphonedb.org/)], highlighting CCL5-CCR1-5 interactions between Temra CD8+ cells and monocytes, NK cells, and other CD4/CD8 T-cell subsets.

FIG. 7. Changes in T-Cell Populations associated with ICI Myocarditis. (A) Identification of peripheral blood CD3+ immune cell clusters across all samples (n=3-6 subjects per cohort). (B) RNA feature plots with canonical markers across CD3+ clusters. (C) Protein expression feature plots of canonical T-cell markers across CD3+ clusters using CITE-seq feature barcoding technology. (D) Heatmap of top differentially expressed genes across clusters. (E) Quantification of CD3+ cell subtypes across clusters and comparison of patient groups. For each cluster, the average fraction of cells from each patient group is shown, after normalization for total CD3+ input cell numbers per patient. Average and SEM are shown for each patient group. Statistical analysis compares groups No ICI, A, B, and C.

FIG. 8. Changes in CD4+ T-Cell Populations with scRNA-seq. Identification of peripheral blood CD4+ immune cell clusters across all samples (n=3-6 subjects per cohort). (B) RNA feature plots with canonical markers across CD4+ clusters. (C) Protein expression feature plots of canonical T-cell markers across CD4+ clusters using CITE-seq feature barcoding technology. (D) Heatmap of top differentially expressed genes across clusters. (E) Quantification of CD4+ cell subtypes across clusters and comparison of patient groups. For each cluster, the average fraction of cells from each patient group is shown, after normalization for total CD4+ input cell numbers per patient. Average and SEM are shown for each patient group. Statistical analysis compares groups No ICI, A, B, and C.

FIG. 9 Changes in Clonal Populations of CD8+ T-Cells Over Time. (A) Sankey diagram showing cluster localization of the top 5 individual clonotypes and late timepoints. (B) Detailed gene expression analysis of top 10 individual expanded TCR clonotypes in the early vs. late timepoints.

### DETAILED DESCRIPTION

Immune checkpoint inhibitors (ICIs) are monoclonal antibodies used to activate the immune system against tumor cells. Despite therapeutic benefits, ICIs have the potential to cause immune related adverse events (irAE) such as ICI-induced myocarditis, a rare but serious side effect with up to 50% mortality in affected patients. Histologically, patients with ICI-induced myocarditis have lymphocytic infiltrates in the heart, implicating T-cell-mediated mechanisms. Current therapeutic options for ICI-induced myocarditis are limited to broad-spectrum glucocorticoids or T-cell suppressive therapies.

In one aspect, this disclosure provides methods that may help in diagnosing and/or treating ICI-induced myocarditis. The ICIs include antibodies, preferably a monoclonal antibody or an antigen-binding fragment thereof, that are used for treating cancer. Certain ICIs are known to selectively bind a regulatory protein present on a T cell, or a ligand for the protein, and thereby activate T-cell cytotoxicity against tumor cells. Examples include antibodies specific to cytotoxic T-cell antigen-4 or programmed death 1 protein (PD-1) or wherein the ligand is programmed death 1 ligand (PDL-1). The ICIs approved by the United States Food and Drug Administration (the FDA) and used for treating patients may include, but are not limited to, Ipilimumab (YERVOY), Nivolumab (OPDIVO), Pembrolizumab (KEYTRUDA), Atezolizumab (TECENTRIQ), Durvalumab (IMFINZI), Avelumab (BAVENCIO), and/or Cemiplimab-rwlc (LIBTAYO). Present methods are suitable for a patient treated with any known ICI.

In this disclosure, a “patient” may be referred interchangeably as a “human subject” or as “subject.” The intended patients are human patients.

The inventors have found that ICI-induced myocarditis is associated with clonal expansion in the peripheral blood of specialized effector CD8+ (cytotoxic) T cells which are distinguishable by expressing myocardial-tropic chemokines CCL3, CCL4L2, CCL4 and CCL5, as well as cytotoxicity markers which may include one or more of the following markers: GZMB, HLA-DRA, KLRB1, KLRF1, and pro-inflammatory interleukins which may include IL-32. These specialized effector CD8+ T cells that associate with ICI-myocarditis may be referred in this disclosure as the Temra CD8+ cells.

Methods according to this disclosure include a method for reducing an immune-related adverse event (irAE) in an immune checkpoint inhibitor (ICI)-treated human subject, wherein the irAE includes ICI-induced myocarditis, and wherein the method may comprise administering to the subject a composition comprising one or more of the following:
i. an inhibitor that antagonizes interaction of chemokine (C-C motif) ligand 5 (CCL5) with at least one of the following receptors: C-C chemokine receptor type 1 (CCR1), C-C chemokine receptor type 3 (CCR3) and/or C-C chemokine receptor type 5 (CCR5);
ii. an inhibitor that antagonizes action of the C-C chemokine receptor type 2 (CCR2);
iii. an antibody binding to and downregulating cytotoxic CD8+T effector cells that express one or more cytotoxicity markers and at least the following myocardial-tropic chemokines CCL3, CCL4, CCL4L2 and CCL5 (Temra CD8+ cells); and/or
iv. an antibody binding to and downregulating monocytes/macrophages that express one or more cytotoxicity markers and either C-C chemokine receptor type 2 (CCR2) or C-C chemokine receptor type 5 (CCR5).

In this disclosure, a human subject may be diagnosed with ICI-induced myocarditis by any method commonly used for diagnosing the condition. Such methods include, but are not limited to, cardiac biomarker troponin I being elevated over the normal range of the general population as detected in the subject's peripheral blood sample, clinical syndrome, negative coronary work-up and/or an imaging diagnosis which may include magnetic resonance imaging and/or positron emission tomography (PET).

In this disclosure, “administering a composition” may comprise administering the composition orally or intravenously, or by any other administration route typically used for delivering a particular inhibitor or an antibody to a patient. The composition may be administered in any therapeutically effective amount, e.g., from about 0.5 mg per a dose to about 300 mg per a dose, suitable for ameliorating the condition to be treated. Other doses may be used as well: 2.5 mg, 50 mg, 75 mg, 150 mg, 500 mg, etc. The composition may be administered at least once daily for one day or during a number of days, e.g., from one day to about 21 days, e.g., 5 days, 7 days, 10 days, 30 days, etc.

In this disclosure reducing an immune-related adverse event that includes ICI-induced myocarditis may be used interchangeably with the term “treating ICI-induced myocarditis.” In this disclosure, “treating ICI-induced myocarditis” means an improvement at least partially of at least some of the patient's symptoms and/or preventing at least some of the patient's symptoms from getting worse.

In some embodiments, reduction or treatment may be determined by measuring initial troponin I levels in the human subject's peripheral blood before the subject is administered the composition comprising the inhibitor and/or the antibody, and then measuring troponin I levels in the subject's peripheral blood again after the composition has been administered at least once, or at least for a number of days, wherein the decrease in troponin I peripheral blood levels, preferably by at least 5% or more in comparison with the initial troponin I levels before the composition is administered, are indicative of ICI-induced myocarditis being treated and/or the irAE being reduced.

Methods according to this disclosure may be practiced by administering to a human subject an inhibitor that antagonizes interaction of CCL5 with one or more of the following receptors CCR1, CCR2, CCR3 and/or CCR5.

Suitable inhibitors may include an antagonist of CCR1, CCR2, CCR3 and/or CCR5. Antagonists include compounds which block signaling through a receptor. Suitable inhibitors include, but are not limited to:
an inhibitor of CCR1 selected from:
   CP-481,715 (N-[(2S,3S,5R)-5-carbamoyl-1-(3-fluorophenyl)-3,8-dihydroxy-8-methylnonan-2-yl]quinoxaline-2-carboxamide, available from Pfizer),
   iMLN3897 (4-(4-chlorophenyl)-1-[(3E)-3-[9-(2-hydroxypropan-2-yl)-5H-[1]benzoxepino[3,4-b]pyridin-11-ylidene]propyl]-3,3-dimethylpiperidin-4-ol, available from Millennium),
   BX471 ([5-chloro-2-[2-[(2R)-4-[(4-fluorophenyl)methyl]-2-methylpiperazin-1-yl]-2-oxoethoxy]phenyl]urea, available from Berlex/Scherring AG), and
   AZD-4818 (2-[2-chloro-5-[3-(5-chlorospiro[3H-1-benzofuran-2,4′-piperidine]-1′-yl)-2-hydroxypropoxy]-4-(methylcarbamoyl) phenoxy]-2-methylpropanoic acid, available from Astra-Zeneca);
an inhibitor of CCR3 selected from:
   SB297006 (ethyl 2-benzamido-3-(4-nitrophenyl) propanoate),
   SB328437 (methyl (2S)-2-(naphthalene-1-carbonylamino)-3-(4-nitrophenyl) propanoate), and
   GW766994 (4-[[[(2S)-4-[(3,4-dichlorophenyl)methyl]morpholin-2-yl]methylcarbamoylamino]methyl]benzamide); and
an inhibitor of CCR5, preferably Maraviroc (monocarboxylic acid amide obtained by formal condensation of the carboxy group of 4,4-difluorocyclohexanecarboxylic acid and the primary amino group of (1S)-3-[(3-exo)-3-(3-isopropyl-5-methyl-4H-1,2,4-triazol-4-yl)-8-azabicyclo[3.2.1]oct-8-yl]-1-phenylpropylamine); leronlimab (PRO 140, a humanized IgG4, kappa monoclonal antibody that recognizes the C-C chemokine receptor type 5, CytoDyn), cenicriviroc (CCR2/5 antagonist), and/or Met-CCL5 (CCL5 antagonist).

Other inhibitors (antagonists) of CCR1, CCR2, CCR3, CCR5 or CCL5 may be also used.

Methods according to this disclosure may be also practiced by administering to a human subject (patient) an antibody that binds to and downregulates cytotoxic CD8+T effector cells that express one or more cytotoxicity markers and at least the following myocardial-tropic chemokines CCL4, CCL4L2 and CCL5 (the Temra CD8+ cells).

Particularly preferred antibodies include a monoclonal antibody specific to receptor CD45RA expressed on the Temra CD8+ cells. Examples of suitable CD45RA antibodies include, but are not limited to, monoclonal antibody clone HI100 or 5H9 (BD Biosciences), or from clone T6D11 (Miltenyi Biotec). These are monoclonal antibodies which react with human CD45RA, a 220 kDa expressed by the Temra CD8+ cells.

The methods according to this disclosure may be practiced by administering one or more of the inhibitors and/or the one or more antibodies either at the time when a patient is still undergoing an ICI treatment, or after the treatment with the ICI has been stopped or has been completed. The compositions may be administered after an ICI-treated subject is diagnosed with ICI-induced myocarditis or even as a preventive measure, especially if the subject has some other complications indicative that the subject may be at high risk for developing ICI-induced myocarditis.

In some embodiments, prior to administering the composition comprising the inhibitor and/or the antibody, the human subject may be tested for the levels of CCL5 mRNA or secreted CCL5 protein in the subject's peripheral blood sample and/or for the levels of the Temra CD8+ cells and/or for the levels of CCR2+ or CCR5+ monocytes/macrophages in the subject's peripheral blood sample. These tests may be conducted by using any method typically used for determining a protein level in a peripheral blood sample and/or for detecting a subpopulation of CD8+ T cells in a peripheral blood sample.

For example, the levels of CCL5 protein may be detected by contacting a peripheral blood sample with an antibody specific for CCL5 protein, for example in ELISA (enzyme-linked immunosorbent assay). In alternative and/or in addition, the levels of CCL mRNA may be tested by real-time PCR, also known as quantitative PCR (qPCR). This test may be conducted on a population of mononuclear cells isolated from the blood sample or on the isolated population of the Temra 8+ cells.

The Temra CD8⁺ cells which indicative of ICI-induced myocarditis, may express at least the following myocardial-tropic chemokines CCL3, CCL4, CCL4L2 and CCL5. The Temra CD8⁺ cells also express CD45RA. These cells also express one or more cytotoxicity markers which may include one or more of the following: GZMB, HLA-DRA, KLRB1, KLRF1 and pro-inflammatory interleukins which may include IL-32. The human subject may be tested for the levels of the Temra CD8⁺ cells in the subject's peripheral blood sample by any method known for sorting T cells. Suitable methods include flowcytometry sorting for CD8⁺ (cytotoxic) T cells and which may then be followed by identifying a subclone of CD8⁺ T cells that express myocardial-tropic chemokines CCL3, CCL4, CCL4L2 and CCL5 by following one or more methods provided in Examples of this disclosure.

In yet another aspect, this disclosure relates to methods for testing and monitoring a patient (human subject) undergoing treatment with at least one ICI. The method may be helpful in detecting and/or monitoring ICI-induced myocarditis at an early stage. These methods may comprise:
1) drawing a first blood sample from the human prior to administering the ICI;
2) drawing a second blood sample from the human subject after administering the ICI;
3) measuring CCL5 protein and/or mRNA levels in the first sample and in the second sample; and/or measuring the Temra CD8⁺ cells in the first sample and in the second sample; and/or
4) measuring CCR2/CCR5 protein and/or mRNA levels in the first sample and in the second sample; and/or measuring a number of monocytes/macrophages expressing one or more cytotoxicity markers and at least the following chemokine receptors (CCR2 or CCR5) in the first sample and in the second sample.

These test methods may be used for addressing whether a human subject is eligible for anti-irAE treatment. Preferably, if the level of CCL5 protein and/or mRNA in the first sample is L1, and the level of CCL5 protein and/or mRNA in the second sample is L2, and when L2 is greater than L1, the human subject is eligible for anti-irAE treatment. At least some of the test methods may be performed by collecting a peripheral blood sample, isolating mononuclear cells from the sample and then detecting the CCL5 protein and/or CCL5 mRNA in the isolated population of CD8+ T cells. At least in some embodiments, the samples may be further tested for protein and/or mRNA levels of at least one of the following: CCL3, CCL4 and/or CCL4L2 in addition to, or instead of testing for CCL5 protein and/or mRNA. The CCL proteins and/or CCL mRNAs may be detected by ELISA and/or by quantitative PCR, respectively.

In yet another aspect, the present disclosure relates to methods for identifying a human subject eligible for ICI-myocarditis treatment, wherein the human subject is undergoing treatment with an immune checkpoint inhibitor (ICI), the method comprising analyzing CD8+ T cell population in a peripheral blood sample of the human subject for presence of Temra CD8⁺ cells expressing myocardial-tropic chemokines: CCL3, CCL4, CCL4L2 and CCL5 and also expressing CD45RA, and wherein when the Temra CD8⁺ cells are present in the blood sample, the human subject is eligible for ICI-myocarditis treatment. This method may include analyzing the CD8+ T cell population during a time period from day 15 to day 35 after initiation of the ICI therapy. However, other time windows, e.g., day one, two and/or at least one day from day one and day ninety, may be suitable as well. The methods may include the time-of-flight mass cytometry (CyTOF) and/or simultaneous single cell RNA-Seq and single cell TCR sequencing. The methods may include collecting a blood sample before the ICI treatment begins, said sample being used as a control sample, and wherein the human subject is eligible for ICI-myocarditis treatment when the presence of the Tempra CD8+ cells is greater in the sample obtained during the ICI-treatment than in the human subject's control sample.

With the development of single-cell phenotyping proteomics and transcriptomics, we now have the ability to study the immune repertoire of various tissues in healthy and disease states at high resolution (16-19). This ability to distinguish between individual cell states is particularly important for conditions that affect immune cell subtypes at a broad level, such as in immune checkpoint inhibition (19). Time-of-flight mass cytometry (CyTOF) is a technology that uses heavy-metal isotopes to stain cells and allows for simultaneous analysis of more than 40 different cellular proteins with minimal compensation (16,17,20,21) and has been validated against flow cytometry for many clinical/research applications including the phenotyping of immune cells in cancer clinical trials (22).

In addition to CyTOF, single-cell RNA sequencing (scRNA-seq) can provide high-throughput transcriptomic information of individual cells and allow for cell-type-specific gene expression analysis and molecular signatures (23). This novel technology, when combined with TCR sequencing, can expand our ability to characterize T-cell repertoires of disease states in humans and animal models (24). The combination of CyTOF, scRNA-seq, and single-cell TCR sequencing can therefore be a powerful combination of technologies to allow for high resolution immune phenotyping of myocarditis in the setting of irAE from ICIs.

Yet, in another aspect, this disclosure relates to a method of treating ICI-induced myocarditis in a human subject, wherein the method comprises monitoring the human subject for a phenotypic shift in the activated Temra CD8+ cell population from an early cytotoxic and pro-inflammatory profile at the onset of ICI-myocarditis to a late exhaustion phenotype expressing known markers of T-cell exhaustion, including KLRG, CX3CR1 and/or LAG3, said exhaustion phenotype develops after ICI-induced myocarditis runs its course for some time period, e.g. 30 days or 60 days.

This disclosure provides a transcriptomic analysis of the Temra CD8+ clones associated with ICI-induced myocarditis, the Temra CD8+ clones having a highly activated and cytotoxic phenotype with expression of cytotoxicity markers such as granzyme/perforin. Longitudinal study provided in this disclosure further demonstrate progression of these Temra CD8+ cells into an exhausted phenotype at about two months after ICI-myocarditis treatment with glucocorticoids.

The profiling of peripheral blood cell in patients with immune-related adverse reaction (IrAE) due to treatment with immune check point inhibitor (ICI) drugs demonstrates the clonal expansion of an effector memory CD8+ T cell population that is likely to play important roles in causing IrAE. These cells express high level of mRNA for the chemokine CCL5 which is well known to be involved in other autoimmune disease processes as a secreted protein. The inventors' finding of the expansion of effector memory CD8+ T cells and their elevated mRNA expression for CCL5 in patients with IrAE are applicable to both diagnostic and therapeutic applications. In diagnostic applications, the abundance of effector memory CD8+ T cells in the blood can serve as a cell-based biomarker of IrAE. The level of secreted CCL5 chemokine in the blood may also be measured as a protein biomarker of IrAE. Furthermore, the mRNA expression level of CCL5 in effector memory CD8+ T cells can be measured as a marker of disease activity. These measurements can serve to diagnose the onset of disease and as a measure of disease activity level for long-term monitoring with repeated measurements.

In therapeutic applications, the effector memory CD8+ T cells and the expression of CCL5 can both be targeted independently or together as a therapy to mitigate the toxic effects of ICI treatment. The targeting of effector memory T cells may be performed by using antibodies against surface markers expressed specifically on effector memory T cells. These antibodies may cause death of effector memory CD8+ T cells due either to Fc-mediated phagocytosis, complement mediated opsonization, or other processes.

The targeting of CCL5 in the blood may be accomplished by using neutralizing antibodies against CCL5 or by blocking the signaling activity of CCR5, the main cell surface receptor for CCL5 that is expressed in monocytes and NK cells. The signaling activity of CCR5 may be abrogated using either blocking antibodies against CCR5 or small molecule chemical that binds and inactivate the signaling activity of CCR5.

This disclosure presents the first comprehensive cellular and transcriptomic profiling of the peripheral blood mononuclear cells of recently diagnosed ICI myocarditis patients and controls using CyTOF, scRNA-seq, and single-cell TCR sequencing. The inventors discovered a unique population of cytotoxic CD8+ T-cells re-expressing CD45RA (Temra) associated specifically with the group of patients with ICI myocarditis compared to control patients with no irAE and those with irAE in other organ systems. This disclosure reports that this population of Temra CD8+ cells were clonally expanded in ICI myocarditis patients, and longitudinal study of these T-cell clonotypes showed an early activated and cytotoxic phenotype followed by a transition to T-cell exhaustion. Additionally, the inventors found an elevated expression of chemokine transcripts in these Temra CD8+ cells that may facilitate homing to the heart, making them attractive candidates for therapeutic targeting.

The invention will now be described in further detail by the way of the following non-limiting examples.

### Example 1. Materials and Methods

To identify immune subset(s) associated with ICI myocarditis, we performed time-of-flight mass cytometry (CyTOF) on peripheral blood mononuclear cells from 40 patients and controls with autoimmune adverse events (irAE) on ICI including four patients with ICI myocarditis. We also utilized multi-omics single-cell technology to immunophenoptype15 patients/controls using single-cell RNA sequencing (scRNA-seq), single-cell TCR sequencing and CITE-seq with feature barcoding for surface marker expression confirmation.

### Study Design and Patient Population

To examine the immune cell subpopulations in patients with ICI-induced myocarditis and compare to patients with other non-myocarditis irAE (e.g. thyroiditis, hepatitis, pneumonitis, etc) with healthy control subjects without cancer and not on ICI, we employed single cell mass cytometry (CyTOF) for quantification of cell populations and markers at the protein level and in parallel 5′ barcoded single-cell RNA-seq (scRNA-seq) with feature barcoding for transcriptomic and immune surface marker analysis. The demographic information for patients and control populations who underwent CyTOF analysis is displayed in Table 1. CyTOF data were obtained from the following patient groups: [i] healthy control patients without cancer and not on ICI treatment, obtained from the Stanford FluPrint dataset (n=23) (25), [ii] Group A: ICI-treated patients without detected irAE (n=5), [iii] Group B: ICI-treated patients with non-myocarditis irAE (n=8), and [iv] Group C: ICI-treated patients with myocarditis (n=4). ICI myocarditis was diagnosed on basis of elevated cardiac biomarkers (troponin I>99th percentile of the general population), clinical syndrome, negative coronary work-up and imaging diagnosis (magnetic resonance imaging and/or PET) (Table 3). In addition to CyTOF data, we obtained single-cell RNA-seq data and TCR sequencing on the following patient populations (Table 2): [i] healthy control patients without cancer and not on ICI treatment obtained from Wilk et al. (n=6) (26), [ii] Group A (n=3), [iii] Group B (n=3), and [iv] Group C (n=3). Disease and control groups were all age and gender matched as available. Only one out of four myocarditis (Group C) patients and two out of eight non-myocarditis irAE (Group B) patients received corticosteroids prior to blood collection.

Our study design allowed us to study changes in immune cell population frequencies as well as transcriptomic changes in these cell populations in the peripheral blood of patients with ICI myocarditis compared to patients with irAE in other organs (e.g. liver, thyroid, and lungs, etc) and healthy controls. All ICI patients had been recently treated with PD-1, PD-L1, or PD-1/CTLA4 antibodies as shown in Tables 1, 2 and 3.

TABLE 1
Demographics of patient cohort undergoing mass cytometry (CyTOF) analysis of peripheral blood.

| | Cytof | | Group B | | |
|---|---|---|---|---|---|
| | Healthy | Group A | ICI + Non- | Group C | |
| | Controls | ICI + No | Myocarditis | ICI + | |
| | No ICI | irAE | irAE | Myocarditis | Total |
| Group Size (n) | 23 | 5 | 8 | 4 | 40 |
| Age | 65.57 | 66 | 65.38 | 80 | 69.24 |
| | (30-90) | (58-87) | (47-84) | (76-85) | (30-90) |
| Gender | Male: n = 17 | Male: n = 4 | Male: n = 6 | Male: n = 4 | Male: n = 31 |
| | Female: n = 6 | Female: n = 1 | Female: n = 2 | — | Female: n = 9 |
| Malignancy | | | | | |
| Lung (Adenocarcinoma) | 0 | 2 | 3 | 3 | 8 |
| Skin (Melanoma) | 0 | 0 | 3 | 0 | 3 |
| Head/neck (Squamous) | 0 | 1 | 1 | 0 | 2 |
| Kidney/Adrenal/Bladder | 0 | 2 | 1 | 1 | 4 |
| Recent | | | | | |
| Immunotherapy | | | | | |
| PD-1 | 0 | 4 | 6 | 3 | 13 |
| PD-L1 | 0 | 1 | 0 | 1 | 2 |
| CTLA-4 | 0 | 0 | 1 | 0 | 1 |
| PD-1 + CTLA-4 | 0 | 0 | 1 | 0 | 1 |
| Diagnosis | | | | | |
| Healthy | 23 | 5 | 0 | 0 | 28 |
| Thyroiditis | 0 | 0 | 2 | 0 | 2 |
| Hepatitis | 0 | 0 | 2 | 0 | 2 |
| Vitiligo | 0 | 0 | 1 | 0 | 1 |
| Colitis | 0 | 0 | 1 | 0 | 1 |
| Pneumonitis | 0 | 0 | 1 | 0 | 1 |
| Pericarditis | 0 | 0 | 1 | 0 | 1 |
| Myocarditis | 0 | 0 | 0 | 4 | 4 |

TABLE 2
Demographics of patient cohort undergoing single-cell
RNA-seq (scRNA-seq) analysis of peripheral blood.

| | | | Group B | | |
|---|---|---|---|---|---|
| | Healthy | Group A | ICI + Non- | Group C | |
| | Controls | ICI + No | Myocarditis | ICI + | |
| | No ICI | irAE | irAE | Myocarditis | Total |
| Group Size (n) | 6 | 3 | 3 | 3 | 15 |
| Age | 4 | 61.67 | 62.33 | 81 | 62.71 |
| | (36-49) | (45-77) | (47-71) | (76-85) | (25-85) |
| Gender | Male: n = 4 | Male: n = 3 | Male: n = 3 | Male: n = 3 | Male: n = 15 |
| | Female: n = 2 | — | — | — | Female: n = 0 |
| Malignancy | | | | | |
| Lung (Adenocarcinoma) | 0 | 1 | 0 | 2 | 3 |
| Skin (Melanoma) | 0 | 0 | 1 | 0 | 1 |
| Head/neck (Squamous) | 0 | 1 | 1 | 0 | 2 |
| Kidney/Adrenal/Bladder | 0 | 1 | 1 | 1 | 3 |
| Recent | | | | | |
| Immunotherapy | | | | | |
| PD-1 | 0 | 2 | 2 | 2 | 6 |
| PD-L1 | 0 | 1 | 0 | 1 | 2 |
| PD-1 + CTLA-4 | 0 | 0 | 1 | 0 | 1 |
| Diagnosis | | | | | |
| Healthy | 6 | 3 | 0 | 0 | 3 |
| Thyroiditis | 0 | 0 | 1 | 0 | 1 |
| Hepatitis | 0 | 0 | 1 | 0 | 1 |
| Vitiligo | 0 | 0 | 1 | 0 | 1 |
| Myocarditis | 0 | 0 | 0 | 3 | 3 |

TABLE 3
Clinical details and demographics of individual patients.

| | | | | | | | | Steroids |
|---|---|---|---|---|---|---|---|---|
| | | | | | | Time from | | before |
| Patient | Phenotype | Gender | Age | Cancer | Drug | onset | | collection? |
| A1 | Negative | Male | 63 | Head/neck | Nivolumab | N/A | | N/A |
| | control | | | | | | | |
| A2 | Negative | Male | 77 | Lung | Durvalumab | N/A | | N/A |
| | control | | | | | | | |
| A3 | Negative | Male | 45 | Adrenal | Pembrolizumab | N/A | | N/A |
| | control | | | | | | | |
| A4 | Negative | Female | 87 | Lung | Cepilimumab | N/A | | N/A |
| | control | | | | | | | |
| A5 | Negative | Male | 58 | RCC | Nivolumab | N/A | | N/A |
| | control | | | | | | | |
| B1 | Thyroiditis | Male | 69 | GU | Ipilimumab, | 3 | months | No |
| | | | | | Nivolumab, | | | |
| | | | | | Pembrolizumab | | | |
| B2 | Grade II | Male | 71 | Head/neck | Nivolumab | 8 | months | Yes |
| | hepatotoxicity | | | | | | | |
| B3 | Vitiligo | Male | 47 | Melanoma | Pembrolizumab | 3 | months | No |
| B4 | Hepatotoxicity/ | Female | 69 | Melanoma | Pembrolizumab, | 5 | months | Yes |
| | colitis | | | | ipilimumab | | | |
| B5 | Thyroiditis | Male | 73 | Melanoma | Pembrolizumab | 8 | months | No |
| B6 | Transaminitis | Female | 58 | Lung | Pembrolizumab | 15 | months | No |
| B7 | Pericarditis | Male | 52 | Lung | Nivolumab | 0 | days | No |
| B8 | Pericardial | Male | 84 | Lung | Pembrolizumab | 2 | days | No |
| | effusion, | | | | | | | |
| | pneumonitis | | | | | | | |
| MCE1/ | Myocarditis, | Male | 76 | Lung | Durvalumab | 1 day | | No |
| MCL1 | troponin I = 10 | | | | | (+repeat | | |
| | ng/mL, diag. | | | | | collection | | |
| | MRI | | | | | at 65 days) | | |
| MCE2 | Myocarditis, | Male | 85 | Lung | Pembrolizumab | 0 | days | No |
| | troponin I = 5 | | | | | | | |
| | ng/mL, diag. | | | | | | | |
| | MRI | | | | | | | |
| MCL3 | Myocarditis, | Male | 82 | Bladder | Pembrolizumab | 1 | month | Yes |
| | troponin I = 4 | | | | | | | |
| | ng/mL, diag. | | | | | | | |
| | MRI | | | | | | | |
| MCE4 | Myocarditis, | Male | 77 | Lung | Pembrolizumab | 0 | days | No |
| | troponin I = 0.6 | | | | | | | |
| | ng/mL, diag. | | | | | | | |
| | PET | | | | | | | |

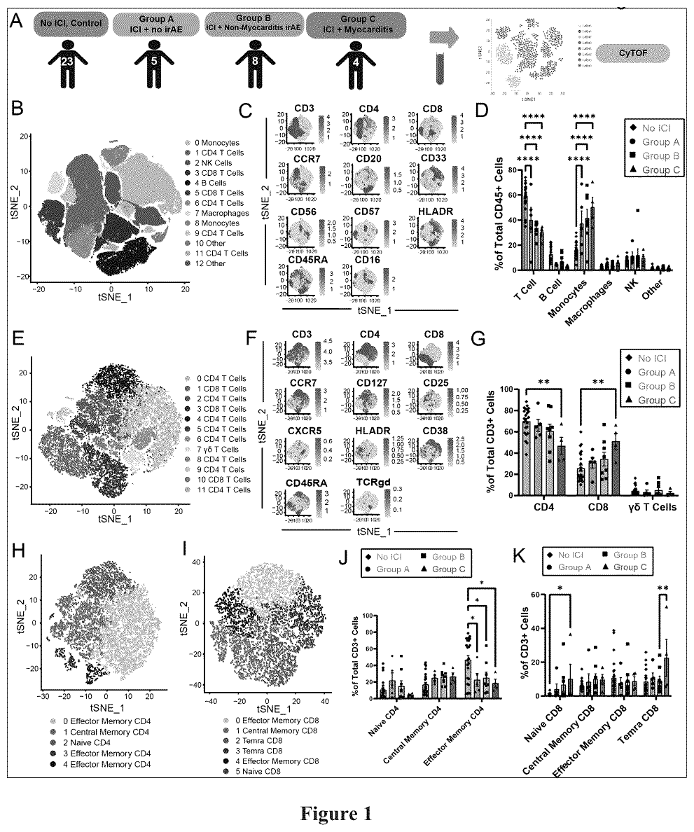

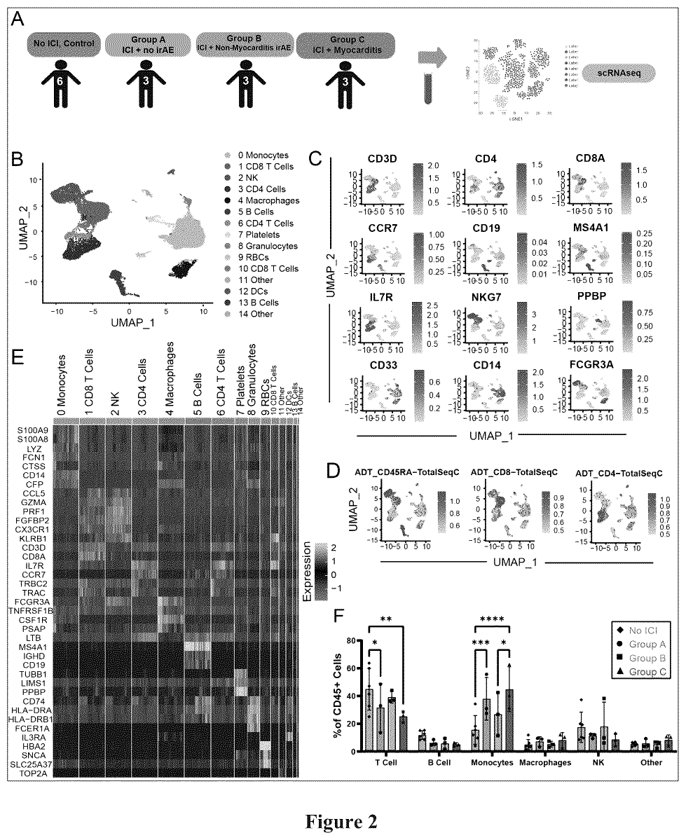

Peripheral blood was drawn from these patients, and peripheral mononuclear cells were isolated for CyTOF (FIG. 1) and scRNA-seq (FIG. 2).

All studies were approved by the Stanford Institutional Review Board and performed in accordance with guidelines on human cell research. Peripheral blood samples were obtained after written informed consent/assent was obtained from participants. Samples were processed and cryopreserved by density gradient separation with Ficoll as described below. Flu database donor CyTOF healthy controls (without cancer and no ICI) were obtained from the Human Immune Monitoring Center (HIMC) and cryopreserved according to standard operating protocols available online at the HIMC website (https://iti.stanford.edu/himc/protocols.html). Processed single-cell transcriptomic data from six health donors were downloaded from the COVID-19 Cell Atlas (https://www.covid19cellatlas.org/#wilk20) hosted by the Wellcome Sanger Institute (26).

### PBMC Collection and Storage

Peripheral blood mononuclear cells (PBMCs) were isolated from EDTA vacutainer blood tubes by density gradient separation with Ficoll-Paque PLUS (GE Healthcare Biosciences, Uppsala, Sweden) within 6 hours of collection. Blood was slowly layered on 5 mL of Ficoll-Paque PLUS in a 15 mL conical tube by pipetting carefully down the side of the tube with a transfer pipette. Tubes were centrifuged at 2000 rpm for 20 minutes with brakes off. PBMCs were aspirated from the density gradient using a 1 mL pipette. The collected cells were washed, and a small volume (10 uL) from each sample was used for counting on a Luna Dual Fluorescence Cell Counter (Logos Biosystems, Annandale, VA). Live and dead cell were distinguished by trypan blue staining. Cells were then pelleted at 1300 rpm for 5 minutes at 4° C. and resuspended in Bambanker freezing medium (GC Lymphotec Inc, Tokyo, Japan) and 4-6×10⁶ cell aliquots were prepared. Cells were frozen overnight at −80° C. @−1° C./min and transferred to liquid nitrogen for cryogenic storage until assay.

### CyTOF/Mass Cytometry Sample Preparation and Staining

Cells were processed according to standard protocols published on the Stanford HIMC website (https://iti.stanford.edu/himc/protocols.html). Briefly, PBMCs were thawed and washed twice in complete medium (RPMI supplemented with Pen-Strep and L-glutamine) and counted using Vi-Cell XR cell viability analyzer (Beckman Coulter, Brea, California). A cell-surface antibody cocktail consisting of pre-conjugated antibodies (Fluidigm, South San Francisco) as well as in-house antibodies (Table 4) were prepared in CyFACS and added to the cells with a 45-minute incubation on ice. All antibody cocktails were filtered using 0.1 um spin filters (Millipore, Darmstadt, Germany) to remove possible antibody aggregates before staining. Cells were washed twice in CyFACS after surface staining and 1:3000 115 In-DOTA Maleimide (5 mg/ml) live/dead stain was added for 30 minutes on ice. Next, cells were fixed in 2% paraformaldehyde (PFA) in PBS at 4° C. overnight. The fixed cells were washed twice in 1× permeabilization buffer (eBioscience, San Diego, CA). After washing three times in CyFACS, cells were stained with Intercalator-Ir (Fluidigm) diluted as per the manufacturer's instructions in 2% PFA in PBS, and incubated for 20 min at room temperature. Finally, the cells were washed twice in CyFACS and three times in MilliQ water. EQ Four Element Calibration Beads from Fluidigm were added as per the manufacturer's directions prior to running. Data were acquired on a Helios mass cytometer (Fluidigm).

TABLE 4
CyTOF panel for phenotypic and functional
analysis of immune cell subsets.

| | | | Conjugated |
|---|---|---|---|
| Metal label | Specificity | Clone | by |
| 102-110Pd | | | |
| 113In | CD57 | HCD57, BioLegend | HIMC |
| 115In | live/dead | | |
| 139La | | | |
| 141Pr | | | |
| 142Nd | CD19 | SJ25C1, Southern BioTech | HIMC |
| 143Nd | CD4 | SK3, BioLegend | HIMC |
| 144Nd | CD8 | SK1, BioLegend | HIMC |
| 145Nd | | | |
| 146Nd | IgD | IA6-2, BioLegend | HIMC |
| 147Sm | CD85j | 292319, R&D Systems | HIMC |
| 148Nd | CD11c | Bu15, BioLegend | HIMC |
| 149Sm | CD16 | 3G8, BioLegend | HIMC |
| 150Nd | CD3 | UCHT1, BD | HIMC |
| 151Eu | CD38 | HB-7, BD | HIMC |
| 152Sm | CD27 | L128, BD | HIMC |
| 153Eu | CD11b | ICRF44, BioLegend | HIMC |
| 154Sm | CD14 | M5E2, BioLegend | HIMC |
| 155Gd | CCR6 | 11A9, BD or G034E3, | HIMC |
| | | BioLegend | |
| 156Gd | CD94 | HP-3D9, BD | HIMC |
| 157Gd | CD86 | IT2.2, BioLegend | HIMC |
| 158Gd | CXCR5 | RF8B2, BD | HIMC |
| 159Tb | CXCR3 | G025H7, Biolegend | HIMC |
| 160Gd | CCR7 | 150503, R&D Systems | HIMC |
| 161Dy | | | |
| 162Dy | CD45RA | HI100, BioLegend | HIMC |
| 163Dy | | | |
| 164Dy | CD20 | 2H7, BioLegend | HIMC |
| 165Ho | CD127 | A019D5, BioLegend | HIMC |
| 166Er | CD33 | P67.8, BD | HIMC |
| 167Er | CD28 | L293, BD | HIMC |
| 168Er | CD24 | ML5, BioLegend | HIMC |
| 169Tm | ICOS | DX29, BD | HIMC |
| 170Er | CD161 | DX12, BD | HIMC |
| 171Yb | TCRgd | B1, BioLegend | HIMC |
| 172Yb | PD-1 | EH12.1, BD | HIMC |
| 173Yb | CD123 | 9F5, BD | HIMC |
| 174Yb | CD56 | NCAM16.2, BD | HIMC |
| 175Lu | HLA-DR | G46-6, BD | HIMC |
| 176Yb | CD25 | M-A251, BD | HIMC |

### CyTOF Data Pre-Processing and Analysis

Data were obtained in the form of .fcs files from the Helios instrument. FCS files were converted into FlowFrame objects using the flowCore package in R (https://bioconductor.org/packages/release/bioc/html/flowCore.html). Individual FlowFrame objects were converted into SingleCellExperiment (SCE) objects for arcsine transformation and the bead normalization using Catalyst R package (https://bioconductor.org/packages/release/bioc/html/CATALYST.html). SCE objects were converted back to FlowFrame objects for live cell gating using the openCyto package (https://bioconductor.org/packages/release/bioc/html/openCyto.html) flowClust.2d automated gating algorithm. Arcsine transformed expression values were imported into Seurat R package (https://satijalab.org/seurat/) for subsequent principle component analysis (PCA) and dimensionality reduction analysis. Each sample began with 250,000 cells, reduced to ˜200,000 cells after live selection, and 7,000 cells per sample were randomly selected for analysis (e.g. tsNE plot generation and cluster identification) for a total of 280,000 cells analyzed (40 samples) in Seurat in the same pipeline as single-cell RNA-seq analysis (see sections on scRNA-seq data analysis below). Unsupervised clustering was then performed using Seurat and major cell populations were named according to well-studied markers in the top rank of differentially expressed markers of each queried cluster. Cell population proportions were calculated and normalized against total CD45+ and CD3+ input cell numbers, and statistical analyses were performed (see Statistics section).

Sample Preparation and Staining for 10×5′ barcoded scRNA-seq with Feature Barcoding and TCR Sequencing

Frozen PBMCs were removed from liquid nitrogen and resuspended in warm RPMI media+10% FBS (@ 37° C.) by adding 1 mL, 3 mL, 4 mL, 8 mL 16 mL media at 30-60 second intervals. Cells were washed and passed through a 40 μm filter, centrifuged @ 300 rcf×5 min and resuspended in PBS with 50 ul 1% BSA and Biolegend Fc Receptor Blocking Solution (Human TrueStain FcX (Cat. No. 422301) for 20 minutes. Afterwards, another 50 μL of antibody mastermix containing Biolegend TotalSeq-C antibodies for staining (Biolegend TotalSeq™-C0063 anti-human CD45RA Antibody Cat. No. 304163; Biolegend TotalSeq™. C0072 anti-human CD4 Antibody Cat. No. 300567; Biolegend TotalSeq™-C0046 anti-human CD8 Antibody Cat. No. 344753) for final manufacture-recommended concentration of 1.0 μg of antibody in 100 μl of staining buffer for every 1 million cells for a total of 10-15 minutes incubation. After incubation, cells were washed 2× by resuspending in 1.5 mL PBS+1% RNase-free BSA (MACS BSA Stock Solution No. 130-091-376) and then 1 mL PBS+0.04% BSA. 10 μL of cell suspension was then taken for counting and appropriate volume of PSA+0.04% BSA was added (200-500 μL) to aim for final target concentration of 1 million cells/mL). Cells were then loaded into Chromium Next GEM Chip G (10×PN 2000177) to achieve 6000-8000 target cell recovery per sample according to the manufacture's protocol.

Library Preparation for scRNA-Seq

All steps from cDNA isolation to library preparation were completed according to 10×'s manufacturing protocols for the Chromium Next GEM Single Cell V (D) J Reagent Kits v1.1. Isolated cDNA was amplified (14 cycles), and then cDNA was allocated for TCR enrichment, feature barcode library generation, and 5′ cDNA library generation. cDNA and library quality was evaluated using the high sensitivity DNA kit on the Agilent 2100 Bioanalyzer.

Sequencing of scRNA-Seq Libraries

Sequencing of all 10× Genomics libraries was performed through Medgenome (Foster City, California). 5′ library, TCR library, and feature barcoding libraries were all sequenced on the Illumina NovaSeq 6000 instrument using 200 cycle kits and run with 2×100 bp for a goal of 20,000 paired reads/cell, 5,000 paired reads/cell, and 5,000 paired reads/cell. Ultimately, 30,000-60,000 mean reads per cell were achieved in each sample, in order to achieve sufficient sampling of genes.

### Single Cell RNA-Seq Data Pre-Processing

Raw single-cell RNA-seq data were processed using 10× Genomics Cell Ranger (4.0.3) to demultiplex the FASTQ reads in order to align them to the human reference genome (GRCh38, v4.0.0, from 10× Genomics) and count the unique molecular identifier (UMI) (through the “cellranger count” function). Individual sample gene expression matrices were loaded into Seurat v4.0.0 R package (https://satijalab.org/seurat/) for further analysis. To filter out poor quality data from cells with high mitochondrial gene expression likely to be dead/damaged cells (59), we removed cells with greater than >15% mitochondrial genes present (60). Additionally, we removed cells for which less than 500 genes were detected. This left us with a total of ˜130,000 cells. We then normalized cell numbers across samples by randomly drawing equal numbers of cells per sample and retained a final number of 28,000 cells for analysis.

### Identification of Cell Clusters

The count matrix containing for each sample was normalized using the ‘NormalizeData’ function in Seurat, which divides the number of UMI for each gene by the total number of UMI in each cell and multiplies by a scale factor of 10,000, followed by adding a pseudocount 1 for each gene and natural-log transformation. Based on normalized gene expression matrix, 2,000 highly variable genes were identified using the ‘FindVariableFeatures’ function with the ‘vst’ method. All patient samples were then integrated into a single Seurat object using the reciprocal PCA (RPCA) multi-modal data integration pipeline in Seurat, through the determination of anchors by the mutual neighborhood requirement (27). ‘ScaleData’ function was then used to scale and center the gene expression matrix. To perform clustering analysis, the first 20 principal components were selected for constructing the shared nearest neighbor (SNN) graph by using ‘FindNeighbors’ function. Louvain clustering algorithm was then used to group the cells into different clusters (61).

### Annotation of Cell Clusters and Data Visualization

We utilized canonical markers of different immune cell subtypes and checked whether these well-studied genes were in the top rank of differentially expressed genes of each queried cluster. The uniform manifold approximation and projection (UMAP) was applied to visualized the single cell transcriptional profile in 2D space based on the SNN graph as described above (62). Feature plots, violin plots, and heatmaps were generated using Seurat's standardized code based on ggplot2 (v3.2.1, R package). To analyze subsets of CD45+ cells (CD3+, CD4+, CD8+ subsets) in detail, we isolated subsets of the CD45+ cell clusters on basis of canonical markers and re-applied the same scaling, finding variable features, and clustering algorithm above to identify sub-clusters of these finer cell subsets. To evaluate for patient-specific batch effects due to technical differences, we evaluated whether clusters containing cells from only a single patient or fraction of patients were present and regressed out unwanted sources of technical variation by implementing ‘ScaleData’ before cell clustering.

### Differential Expression Analysis

We identified differentially expressed genes in different clusters using the ‘FindAllMarkers’ function (using default parameters; min.pct=0.25, logfc.threshold=0.25) in Seurat to help identify cluster and sub-cluster identities. We also utilized the ‘FindMarkers’ function for differential expression of analysis between patient groups (Group A vs B vs C) and between time conditions (early vs late timepoints for myocarditis patient MCE1-see Results section).

### T Cell Receptor Data Analysis

Raw T cell receptor (TCR) sequencing data were processed using 10× Genomics Cell Ranger (v4.0.3). In summary, the algorithm aligned FASTQ reads to human GRCh38 V (D) J reference genome (v4.0.0, from 10× Genomics) by using the “cellranger vdj” function, resulting in the assembly of V (D) J sequences and clonotypes (36). On average, 47,975 read pairs were detected per cell in each patient sample. On average, 83.2% (and 95.7%, respectively) of CD3+ cells were associated with at least one productive TRA (TRB, respectively) rearrangement, and 79% of the cells were associated with productive V-J spanning pairs, and of those, 85% were associated with unique ab TCR clonotypes. The filtered contig annotations, which contained high-level annotations of each high-confident cellular contig was used. Using customized Python code, the clonotype ID and cell barcode IDs within each unique sample were paired and inputted into Seurat for visualization of individual T-cell clonotypes on the UMAPs.

### Cluster Analysis of Clonally Expanded T-Cells

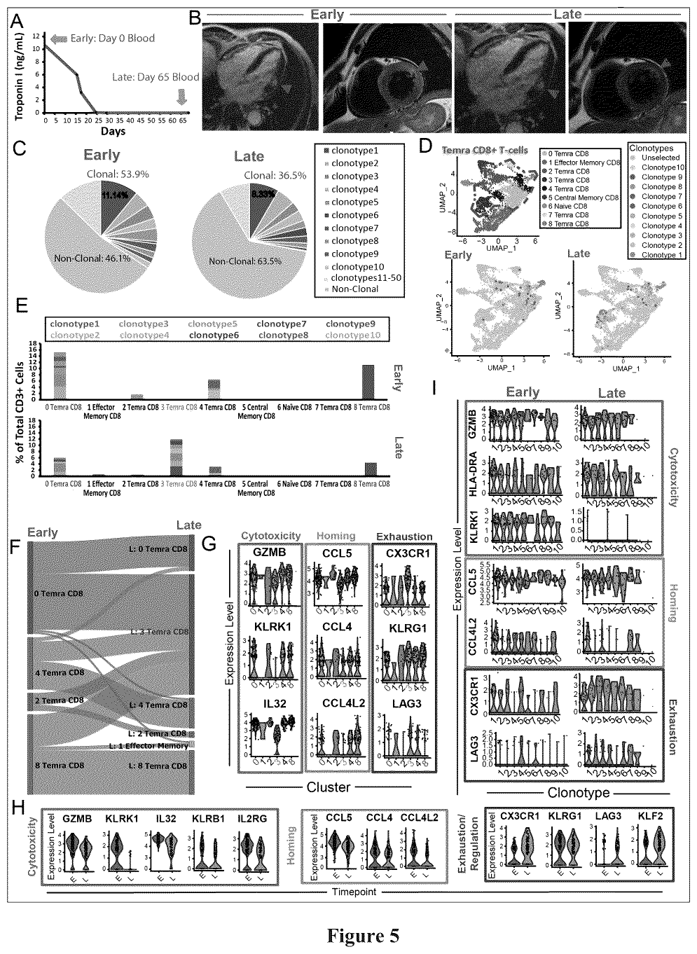

On average, we could detect 2,254 unique ab TCR clonotypes in each patient (ranging from 399 to 8197 among all patient samples). For the total 27,057 unique clonotypes detected in all patient samples, clonotype size ranged from 1 to 578 cells (up to 11.14% of all CD3+ cells for a single clonotype in a single sample). To quantify clonotypes across clusters, we used Seurat to interrogate the clonotype frequency within each cluster and plot the frequencies in bar graphs. To visualize clonotypes on the UMAPs, we utilized our custom Python code to correlate cell barcodes with individual clonotype IDs within each sample and mapped them onto the UMAPs in Seurat. We normalized the clonotype frequency to the total input CD45+ input cell number for each sample and plotted the top 50 clonotypes within each patient group. To interrogate clonotypes across timepoints (FIG. 5), we correlated clonotypes for the same patient across timepoints using the unique ab chains and assigned them a clonotype ID, then plotted them in Seurat. To generate Sankey diagrams of clonotype lineage tracing over time, we tracked the same clonotype IDs across the two different timepoints. Differential expression gene analysis was performed on the top 10 most frequent clonotypes in the first timepoint compared to the same clonotypes in the second timepoint and significant genes were plotted using the violin plot function in Seurat.

### Ligand-Receptor Expression and Cell Interactions

We examined the interaction of Temra CD8+ cells in our scRNA-seq patient CD45+ dataset based on the expression level of ligand-receptor pairs using the CellPhoneDB database (63) (https://www.cellphonedb.org/), a publicly available repository of curated receptors, ligands and their interactions which uses computational approaches to identify biologically relevant interacting ligand-receptor partners from scRNA-seq data. The database takes into account the subunit architecture of both ligands and receptors, as well as expression levels of ligands and receptor within each sample and uses a statistical framework and empirical shuffling to predict which ligand-receptor pairs may be significant between cell types within the specified scRNA-seq dataset. We inputted our CD45+ dataset and received output in the form of a.csv file containing significant predicted ligand receptor interactions on basis of mean expression level in our cell type of interest (Temra CD8+ cells) with mean expression level in the interacting cell types (all other cell types within our CD45+ dataset). This generated a null distribution for each ligand-receptor pair in each pairwise comparison between cell types, for significant p value<0.01. The top 30 ligand-receptor interactions ranked by weighted average mean expression value of the ligand-receptor pair were graphed on a dot plot through ggplot2 in R.

### Data and Materials Availability

CyTOF data has been uploaded onto the Mendeley Data repository at doi: 10.17632/cm8pxdt5 pm.1. All single-cell RNA-seq, single-cell TCR-seq, and CITE-seq data in this paper have been uploaded onto the NCBI Gene Expression Omnibus (GEO) data repository at accession number GSE180045.

### Statistical Methods

Statistical comparisons between cell subgroups were performed within each patient group using standard statistical methods. Specifically, averages were calculated within each patient group and standard error measurements were calculated within each group. Two-way ANOVA along with Tukey's multiple comparisons test were used to determine significant cell proportion differences between patient groups. Correction for multiple comparisons using statistical hypothesis testing was performed, and a significance threshold of p<0.05 (95% confidence interval) was set.

### Example 2. Results

Using these complementary approaches, we found an expansion of cytotoxic CD8+T effector cells re-expressing CD45RA (Temra CD8+ cells) in ICI myocarditis patients compared to controls. Using TCR sequencing, we demonstrated that these CD8+ Temra cells were clonally expanded in myocarditis patients compared to controls. Transcriptomic analysis of these Temra CD8+ clones confirmed a highly activated and cytotoxic phenotype with expression of cytotoxicity markers such as granzyme/perforin. Longitudinal study demonstrated progression of these Temra CD8+ cells into an exhausted phenotype two months after treatment with glucocorticoids. Differential expression analysis demonstrated elevated expression levels of proinflammatory chemokines (CCL4/CCL4L2/CCL5) in the clonally expanded Temra CD8+ cells. Ligand-receptor analysis of the Temra CD8+ cells implicated their regulation of other inflammatory cells such as monocytes and NK cells.

### Example 3. Analysis of Immune Cell Populations in ICI Myocarditis Using CyTOF Reveals Cytotoxic Temra CD8+ Expansion

We analyzed peripheral blood mononuclear cells (PBMCs) from 40 patients categorized into the four patient groups as described in the Methods section (No ICI Group-Healthy patients without cancer and not on ICI from the Stanford FluPrint Database, n=23 (25); Group A-Patients on ICI without irAE, n=5; Group B-Patients on ICI with non-myocarditis irAE, n=8; Group C-Patients on ICI with ICI myocarditis, n=4) (Table 1 and Table 3). All patients were recruited according to a protocol approved by the Stanford Institutional Review Board. A total of 280,000 cells from the peripheral blood of these patients were analyzed using CyTOF (FIG. 1A) with a panel of 33 canonical immune surface markers (Table 4). Unsupervised clustering revealed 12 distinct clusters comprised of all expected immune cell populations including T cells, B cells, monocytes, macrophages, and natural killer cells (FIG. 1B). Feature plots showed robust expression of major immune canonical markers within the expected immune subsets (FIG. 1C). Clusters of cell populations were marked according to differentially expressed markers of each queried cluster as well as established canonical markers of each immune subset. Quantification of immune cell distributions across cell clusters showed a relative reduction in circulating T-cells in the ICI-treated groups (Groups A-C) compared to the “No ICI” control group (FIG. 1D). Additionally, there was an increase in circulating monocytes in the ICI-treated patients (Groups A-C), with the ICI myocarditis patients (Group C) showing the largest increase relative to the “No ICI” group. We then subsetted CD3+ cells and performed unsupervised clustering, found subclusters of CD3+ cells (FIG. 1E), identified canonical CD3+ gene markers (FIG. 1F), and quantified CD3+ subtypes as a proportion of the total CD3+ input number per sample (FIG. 1G). We observed a significant increase in CD8+ cells in the ICI-myocarditis group (Group C) patients compared to the non-ICI treated group, but no significant difference between the CD4+ and CD8+ populations between ICI-treated groups A/B/C. We then further selected subsets of cells from the CD3+ population—the CD4+ (FIG. 1H) and CD8+ populations (FIG. 1I). Subsequently, we used differential expression analysis and canonical markers of CD4+ and CD8+ subsets to identify cell populations. Quantification of the CD4+ subtypes did not show significant trends among the ICI-treated patient groups (Group A/B/C) (FIG. 1J), but quantification of CD8+ subtypes showed a significant increase in the Temra CD8+ (T-effector cells re-expressing CD45RA) CD8+ sub-population in the ICI myocarditis group (Group C) compared to both ICI-treated control groups (Groups A and B) as well as the non-ICI treated group (FIG. 1K). These results demonstrate that ICI-myocarditis (Group C) is associated with a significant increase in Temra CD8+ cells in the peripheral blood compared to ICI-treated patients without side effects (Group A) and with non-myocarditis irAE (Group B).

### Example 4. Global Analysis of Immune Cell Populations in ICI Myocarditis Using scRNA-Seq Corroborates CyTOF Results

To confirm our results from CyTOF studies and gain further insights into the molecular characteristics of circulating immune cell subsets that are differentially expanded in ICI-myocarditis patients, we performed scRNA-seq analysis on PBMCs from 15 patients (Table 2). This included healthy control patients without cancer who are not on ICI, n=6 (26) and ICI-treated patients whose blood we collected using a Stanford IRB-approved protocol-Group A, n=3; Group B, n=3; Group C, n=3. We obtained high-quality transcriptomes from a total of 130,000 cells (FIG. 2A) and performed integration and batch correction of the data using the reciprocal PCA (RPCA) multi-modal data integration pipeline in Seurat (27). We observed 14 distinct clusters comprising all expected immune populations, including T-cells, B-cells, monocytes, macrophages, NK cells, dendritic cells and granulocytes (FIG. 2B). Gene expression feature plots showed presence of major immune canonical markers within the expected immune subsets (FIG. 2C). For genes that exhibit low level of expression at the RNA level (e.g. CD45RA, CD4), feature barcoding using oligonucleotide-tagged antibodies to selected surface receptors (e.g. CD4, CD8, CD45RA) was performed simultaneously on cells captured for scRNAseq profiling to correlate RNA expression level with protein expression level in the same single cell (FIG. 2D; Table 5).

TABLE 5
Feature Barcoding (CITE-seq) Antibodies

| Antibody | Clone | Cat. No. |
|---|---|---|
| Biolegend TotalSeq ™-C0063 | HI100 | 304163 |
| anti-human CD45RA Antibody | | |
| Biolegend TotalSeq ™-C0072 | RPA-T4 | 300567 |
| anti-human CD4 Antibody | | |
| Biolegend TotalSeq ™-C0046 | SK1 | 344753 |
| anti-human CD8 Antibody | | |

Clusters of cell populations were marked according to differentially expressed markers as well as known canonical markers of each queried cluster (FIG. 2E). Similar to the CyTOF data, a relative increase in monocytes and a reduction in circulating T-cells were found in the ICI-treated patients (Groups A-C) compared to the “No ICI” control group (FIG. 2F). Additionally, there was an increased expansion of circulating monocytes in the myocarditis (Group C) patients compared to the other groups.

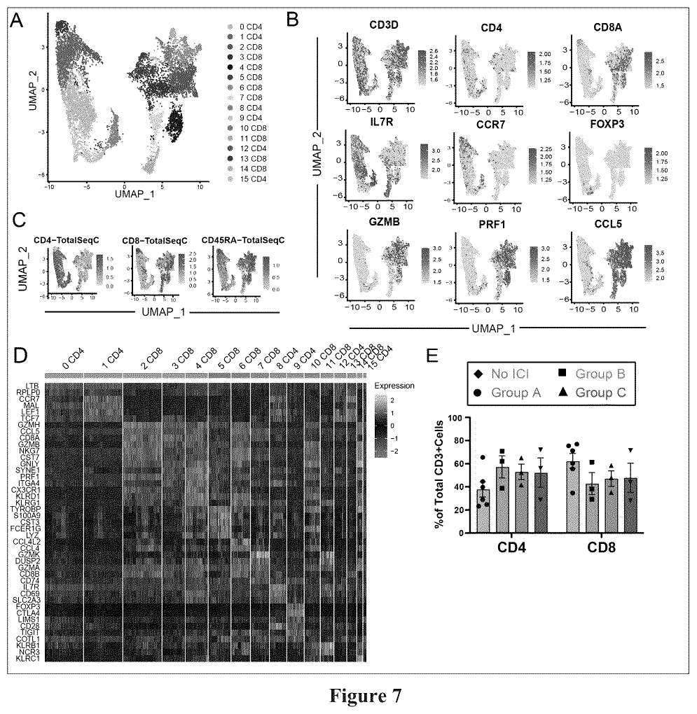

We then subsetted CD3+ cells and performed unsupervised clustering to find differentially expressed genes for each cluster (FIG. 7A-D) and quantified the frequency of each CD3+ subcluster (FIG. 7E). We found no significant differences between the proportion of CD4+ and CD8+ cell populations between the patient groups.

### Example 5. Confirmation of Myocarditis-Associated Temra CD8+ Expansion by scRNAseq

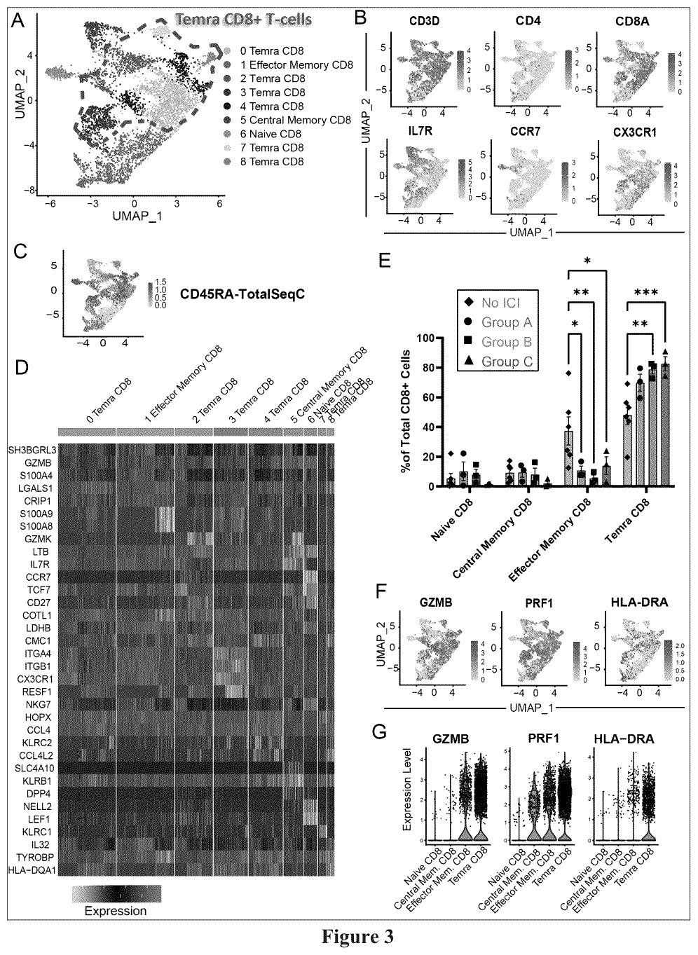

To evaluate changes in CD8+ subpopulations in ICI myocarditis compared to control groups, we subsetted CD8+ cells from our dataset and performed unsupervised clustering on this population (FIG. 3A). Feature plots showed distinct RNA expression patterns of genes such as CCR7, and IL7R that discriminate between different CD8+ subpopulations (FIG. 3B). The presence of the surface marker CD45RA was also detected by feature barcoding using oligo-conjugated antibodies (FIG. 3C). Based on the differential expression of these and the top canonical markers of CD8+ cells, we identified the major CD8+ subpopulations including naïve, central memory, effector memory, and Temra CD8+ cells (FIG. 3D). Quantification of the frequency of CD8+ subtypes demonstrated a relative increase in Temra CD8+ population in the myocarditis group (Group C) compared to the other ICI-treated control groups (Group A/B) that did not reach statistical significance, but a statistically significant increase in the Temra CD8+ population was found when compared to the non-ICI treated group (FIG. 3E). These results, like the CyTOF data, demonstrate that ICI myocarditis may be associated with an increase in Temra CD8+ cells in the peripheral blood. Furthermore, these Temra CD8+ cells expressed high levels of cytotoxicity markers including granzyme B (GZMB) and perforin (PRF1) as well as the activation marker, HLA-DRA, relative to the other CD8+ cell types (FIGS. 3F and 3G). This finding suggests a cytotoxic role for these Temra CD8+ cells.

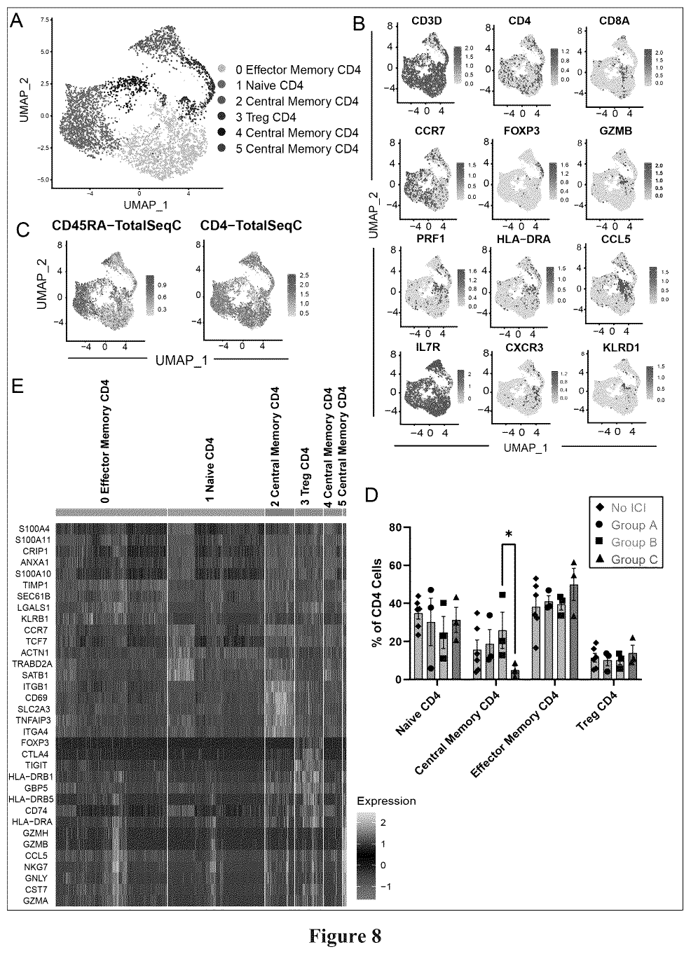

To assess whether a unique subpopulation of CD4+ cells may be present in ICI-myocarditis, we evaluated the CD4+ subpopulations in a fashion similar to the CD8+ subpopulation (FIG. 8). However, beyond a decrease in the central memory CD4+ cell population in the ICI myocarditis group, we did not detect any other significant changes in cell proportions between the patient groups.

### Example 6. Single-Cell TCR Sequencing Reveals Myocarditis-Associated Clonal Expansion of Cytotoxic Temra CD8+ Cell Clusters

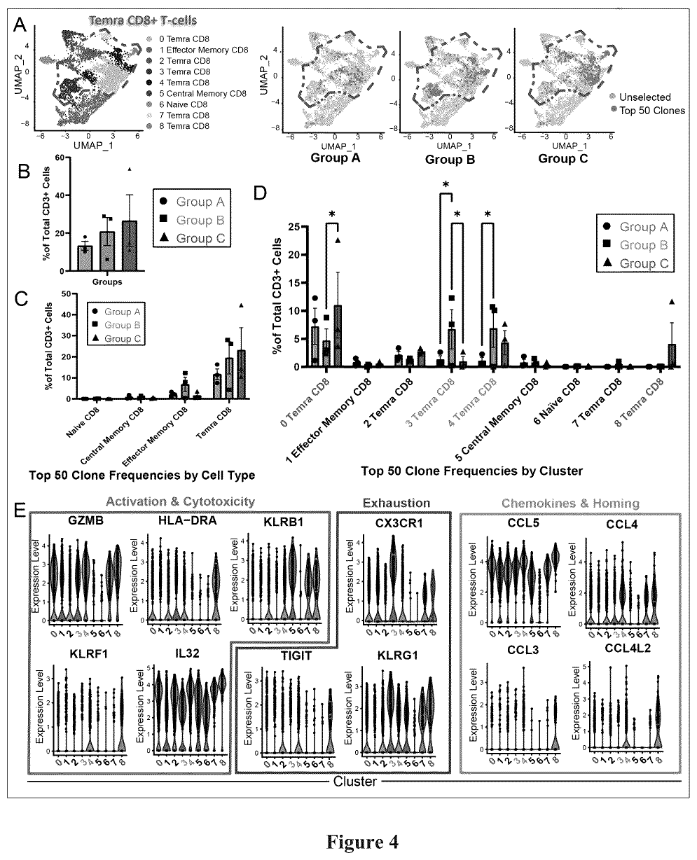

Clonal expansion of T-cells occurs after T-cell activation, and has been thought to play a role in autoimmunity (28). In order to interrogate the clonal identities of the expanded T-cells in our ICI myocarditis cohort compared to the other control patient groups, we performed single-cell TCR sequencing on our patient groups A, B, and C using the 10× Genomics V (D) J single-cell TCR sequencing assay to evaluate productive V-J spanning pairs within each patient sample. Most of our CD3+ cells express αβ TCRs (85% of sequenced TCRs). For each patient, we identified the top 50 clonotypes and visualized their gene expression signature across patient groups A, B, and C. We found an expansion of the top 50 clonotypes in ICI myocarditis patients (Group C) compared to the control (Group A) and non-myocarditis (Group B) populations (FIG. 4A, B). Furthermore, most of these clonotypes were localized to the Temra CD8+ clusters (FIG. 4A). Quantification of the top 50 TCR clonotypes as a fraction of total CD3+ cells showed an enrichment of clonotypes within the Temra CD8+ cell cluster in myocarditis patients (Group C) compared to other groups (FIG. 4C). We further analyzed the abundance of top 50 TCR clonotypes within subclusters of CD8+ cells and found preferential expansion of TCR clonotypes in subclusters 0 and 8 of Temra CD8+ cells (highlighted in red, FIG. 4D). In contrast, subclusters 3 and 4 of Temra CD8+ appear to be preferentially expanded in patients with non-myocarditis irAE (group B). These findings indicate the presence of transcriptionally distinct populations of Temra CD8+ cells in ICI myocarditis patients compared with Temra CD8+ cells from patients with non-myocarditis irAE, suggesting the likelihood that a heart-specific inflammatory trigger is present in ICI myocarditis patients.

To evaluate transcriptomic differences between different CD8+ cell clusters, we performed differential gene expression analysis (FIG. 4E) and visualized the results with violin plots to find that clusters 0, 3, 4, and 8, particularly cluster 8, showed a relatively high expression of activation and cytotoxicity markers (GZMB, HLA-DRA, KLRB1, KLRF1) and pro-inflammatory interleukins (IL-32). Myocarditis-associated cluster 8 also showed an increased expression of myocardial-tropic chemokines CCL3/CCL4L2/CCL4/CCL5 (29,30). Interestingly, the receptors for these chemokines, specifically CCR1 and CCR5 have been shown to be robustly expressed in failing and non-failing hearts (30), and CCR5 in particular has had an established role in myocarditis associated with Chagas disease and cardiac autoimmunity (29,31). In contrast to the myocarditis-associated Temra CD8+ clusters (e.g., cluster 0 and 8), the non-myocarditis-associated Temra CD8+ cluster (e.g. cluster 3 and 4) showed higher expression levels of T-cell exhaustion markers (CX3CR1, KLRG1). These transcriptomic findings suggest a phenotypic difference between the myocarditis-associated clusters relative to the non-myocarditis associated clusters in terms of stage of T-cell activation. Namely, Temra CD8+ cells from non-myocarditis irAE patients exhibit increased markers of T-cell exhaustion compared to myocarditis patients and the myocarditis-associated clusters expressing higher levels of chemokines shown to interact with chemokine receptors expressed in cardiomyocytes (30).

### Example 7. Activated and Expanded Temra CD8+ Clones in Myocarditis Transition from an Early Cytotoxic Phenotype to a Late Exhaustion Phenotype Over Time

In order to evaluate the time evolution of clonally-expanded T-cells in ICI myocarditis, we longitudinally analyzed PBMCs from one patient with MRI-confirmed myocarditis at early (t=0 days) and late (t=65 days) timepoints (FIG. 5A). This patient received glucocorticoids (60 mg/day of prednisone) for treatment of his myocarditis on the first day of his diagnosis (after blood collection) and underwent a successful four-week steroid taper with resolution of his cardiac biomarkers (troponin I, measured in ng/ml) by day 25. Cardiac MRI also confirmed the initial presence of late gadolinium enhancement (LGE) with increased T2 signaling suggestive of myocardial edema at day 0 which resolved by day 65 (FIG. 5B). Clonal analysis of CD3+ cells in his peripheral blood in the early and late time points showed an oligoclonal expansion of top 50 TCR clonotypes (53.9% of all CD3+ cells in peripheral blood) at the time of diagnosis of myocarditis which continued to persist (36.5% of CD3+ cells) at the later timepoint. Furthermore, the clonotype with the biggest expansion (defined as “clonotype 1”) accounted for 11.14% of all T-cells in the peripheral blood of this patient at initial diagnosis and decreased only slightly to 8.33% by day 65 despite steroid treatment (FIG. 5C). This data supports the persistence of clonally expanded Temra CD8+ cells in myocarditis patients even after clinical indices of inflammation have resolved.

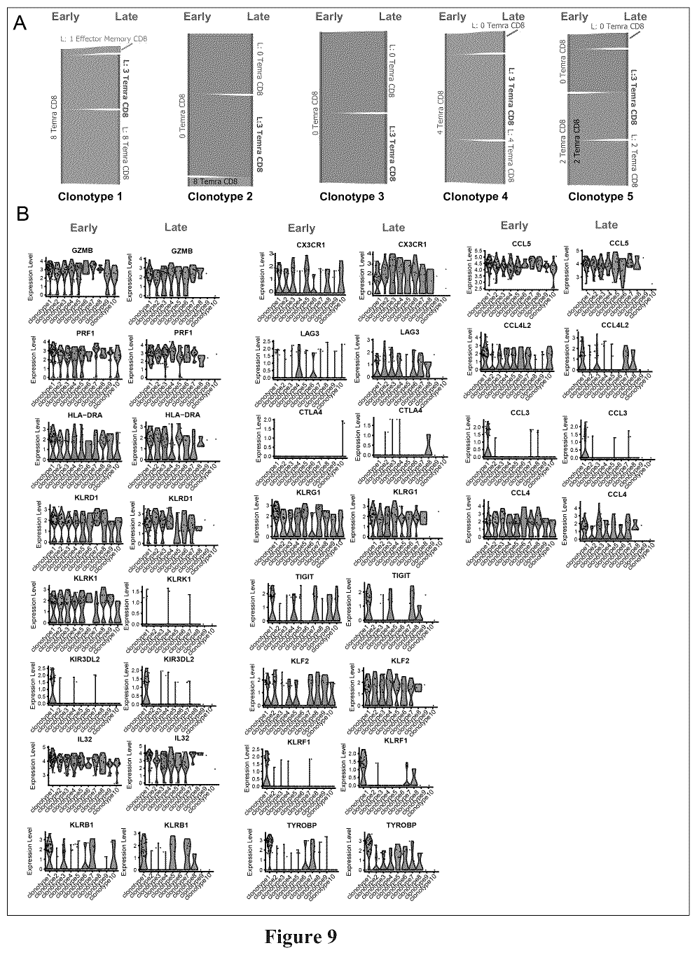

To assess the transcriptomic changes in the top 10 expanded TCR clonotypes in this patient over time, we tracked the cellular identity of the top 10 expanded TCR clonotypes from initial diagnosis to day 65. We found that these top expanded clonotypes were localized in clusters 0 and 8 at the time of diagnosis but translocated to cluster 3 at day 65 (FIG. 5D). This was confirmed quantitatively (FIG. 5E) and by Sankey diagrams that trace the lineage destination of the clonotypes from the early to late timepoints (FIG. 5F, FIG. 9A).

To better understand the transcriptional differences between clusters 0, 3, and 8, we performed differential expression analysis on the top 10 clonotypes across the clusters. Among the top differentially expressed markers of each cluster, the early timepoint-associated clusters 0 and 8 demonstrated relatively high expression levels of cytotoxicity markers (GZMB, KLRK1, IL32) and homing-associated chemokine genes (CCL5, CCL4 and CCL4L2) compared to the late timepoint-associated cluster 3 (FIG. 5G). Notably, the expression of KLRK1, which encodes the activating cell surface receptor NKG2D known to be expressed on immune cells in rheumatoid arthritis and autoimmune colitis (32), was highly expressed in clusters 0 and 8 but not in cluster 3. Cluster 3, in contrast, demonstrated relatively high expression levels of T-cell exhaustion markers (CX3CR1, KLRG1, and LAG3) compared to clusters 0 and 8. Notably, the expression of the immune checkpoint, LAG3, generally responsible for suppressive immune responses, was high in cluster 3 but virtually not expressed in clusters 0 and 8.

Next, we assessed the differential expression of genes between the early and late timepoints in the top 10 clonotypes and similarly found a relative decrease in T-cell cytotoxicity/activation genes (GZMB, KLRK1, IL32, KLRB1, IL2RG), decrease in homing chemokine gene expression (CCL5, CCL4, CCL4L2), and increase in T-cell exhaustion/regulatory genes (CX3CR1, KLRG1, LAG3, KLF2) in the late timepoint compared with early timepoint (FIG. 5H). Individual gene expression analysis of the top 10 clonotypes (FIG. 5I, FIG. 9B) demonstrated a similar pattern in decreased expression of cytotoxicity/homing genes and increase in T-cell exhaustion and regulatory markers in the late compared to early timepoints.

Overall, these findings suggest that early cytotoxic transcriptomic programs (elevated GZMB, KLRK1, KLRB1) in these myocarditis-associated clonally-expanded Temra CD8+ T-cells become attenuated with time. Concordant with these changes, an increase in T-cell exhaustion markers (KLRG1, CX3CR1, LAG3, KLF2) was noted in the same Temra CD8+ cells at the late timepoint. Additionally, there is an early robust expression of chemokines (CCL4, CCL5, CCL4L2) that are involved in the recruitment of immune/inflammatory cells to the heart which decreases over time, giving clues to the organ-specific nature of cellular inflammatory programs upregulated with ICI myocarditis.

### Example 8. Ligand-Receptor Analysis of Myocarditis-Associated T Cell Populations Exhibits Interactions with Other Immune Cell Types Through Cardiotropic Chemokines

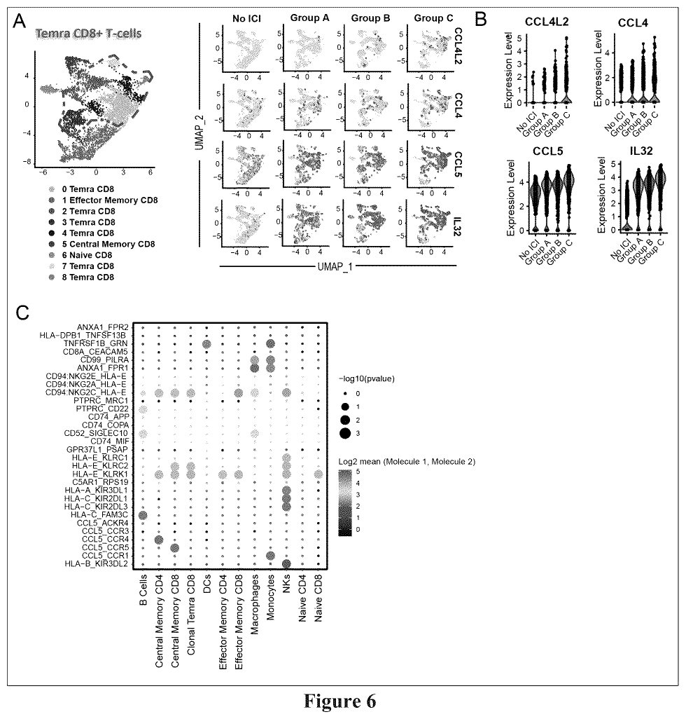

In order to investigate functional interactions in our Temra CD8+ population of interest with other cell types, we performed differential expression analysis across patient groups A-C for the Temra CD8+ cells, as well as across the other CD8+ and CD4+ subtypes. For Temra CD8+ cells, the top differentially expressed genes across the patient groups are shown in the feature plots in FIG. 6A, highlighting once again the upregulation of chemokines CCL4L2, CCL4, CCL5 as well as the pro-inflammatory cytokine IL-32 in the myocarditis cohort (Group C) compared to the other control groups. Quantification of log-fold gene expression levels confirmed this finding (FIG. 6B).

To examine the potential interactions in vivo of expanded Temra CD8+ cells with other immune cells we used the CellPhoneDB algorithm (33) to identify biologically relevant interactions between known ligand-receptor pairs. CellPhoneDB, a tool which uses a combination of computational approaches and a publicly available database of curated receptors and ligands, allows for the prediction of potential ligand-receptor interactions within a scRNA-seq dataset (FIG. 6C). We applied this algorithm to our CD45+ dataset and identified statistically significant interactions between ligands expressed by our Temra CD8+ cells with circulating monocytes (via the crosstalk between CCL5-CCR1) and NK cells (via the HLA-B-KIR3DL2 interactions), as well as many other subtypes of CD4/CD8 T cells (via the CCL5-CCR5 interaction). These data support our hypothesis that CCL5 may be an important chemokine in the pathogenesis of these Temra CD8+ cells, permitting their interactions with other players in the innate and adaptive system to recruit them to target organ sites—in particular, through CCR5 expressed in the heart—in our patient cohort.

### Example 9. Discussion

Clonal cytotoxic Temra CD8+ cells are significantly increased in patients with ICI myocarditis, and have unique transcriptional changes, including upregulation of chemokines CCL4/CCL4L2/CCL5, which may serve as attractive diagnostic/therapeutic targets for reducing life-threatening cardiac immune-related adverse events in ICI-treated cancer patients.

In this study, we present the first comprehensive profiling of the transcriptome of peripheral blood of patients with ICI myocarditis in comparison to other non-myocarditis irAEs using high resolution single-cell proteomic and transcriptomic profiling techniques. Using both high dimensional mass cytometry (CyTOF) for proteomic data and single-cell RNA-seq for transcriptomic data, we found an expanded population of cytotoxic Temra (effector) CD8+ cells in patients with ICI myocarditis compared to all other control groups, including patients without irAEs and patients with non-myocarditis irAEs. Furthermore, we observed that these cytotoxic Temra CD8+ cells are clonally expanded in the blood of myocarditis patients compared to the other patient control groups. They also appear to express increased levels of cytotoxicity markers (GZMB, PRF1, KLRK1, KLRB1, KLRF1), pro-inflammatory interleukins such as IL-32, and pro-inflammatory chemokines (CCL5/CCL4/CCL4L2) with an affinity towards chemokine receptors known to be expressed in the heart (CCR1/CCR5) (30). We believe that these cytotoxic expanded Temra CD8+ cells expressing increased levels of cardio-tropic chemokines play a critical role in the pathogenesis of ICI myocarditis.

Due to its fulminant nature with the risk of heart failure, arrhythmias and death (34), ICI myocarditis remains one of the most feared complications of ICI therapy. Because an understanding of the mechanism of pathogenesis as well as targeted therapeutics for this devastating complication are lacking, our study should provide useful insights into key immune cell subsets and transcriptomic profiles implicated in ICI myocarditis compared to irAE in other organs, thus providing several potential diagnostic and therapeutic targets for this disease.

Our data showing an expansion of cytotoxic CD8+ T-cell populations in ICI-induced myocarditis patients is consistent with prior human and mouse studies in the field. Post-mortem cardiac muscle histopathology of ICI myocarditis patients have previously demonstrated intense patchy lymphocytic infiltrates within the myocardium of CD8>CD4 T-cells (34). Genetic knockout of the immune checkpoint PD-1 in the MRL-Pdcd-/-mouse model have also demonstrated spontaneous CD8>CD4 lymphocytic infiltrates in the hearts by 4-6 weeks of age (14). However, no prior studies have delineated these cytotoxic T-cells as Temra CD8+ cells, as well as demonstrated their increased expansion in the peripheral blood of ICI myocarditis patients compared to the blood of patients with non-myocarditis irAE. Interestingly, Temra CD8+ cells have previously been shown to be expanded in the peripheral blood of patients with other types of autoimmune disease—for example, in rheumatoid arthritis (35). However, they have never previously been shown to play a role in myocarditis. Additionally, our in-depth transcriptomic profiling of these Temra CD8+ cells, including the finding of their increased expression of the chemokines CCL5/CCL4/CCL4L2 is novel. Recently published data in mice with TnI-directed autoimmune myocarditis were also found to have elevated levels of expression of CCL3, CCL4 and CCL5 and their chemokine receptors CCR1/CCR2/CCR5 in their hearts (8), suggesting a potential mechanism for T-cell homing to the heart. However, this has not been previously shown in circulating lymphocytes in humans.

In contrast to prior studies reporting increased Th17 effector cell activity in the hearts of patients with ICI myocarditis (8), we did not see evidence for increased Th17 effector cell activity in the peripheral blood of our patients, suggesting that there may still be differences between T-cell activity in the heart vs blood in ICI myocarditis. Another study reported increased Th17 cell activity in the blood of patients with autoimmune myocarditis; however, these patients had not been treated with immune checkpoint inhibitors, suggesting differential mechanisms of pathogenicity between these varying types of myocarditis (6). Although our study focused on the peripheral blood of ICI myocarditis patients rather than biopsied heart tissue, we were able to gain proteomic and transcriptomic single-cell data in a larger cohort of ICI myocarditis patients and make direct comparisons to several key control groups-healthy patients not on ICI, patients on ICI who experienced no irAE (Group A), and patients on ICI who experience non-myocarditis irAE (Group B). In our study, we found a clonal Temra CD8+ cell expansion and elevated expression levels of heart-tropic chemokines CCL5/CCL4/CCL4L2 previously shown to play a role in myocardial inflammation (29) can be observed even in the peripheral blood, highlighting them as important peripheral biomarkers in the disease. Furthermore, our ligand-receptor analysis supported the interactions of these Temra CD8+ cells with other players in the innate and immune adaptive immune system (monocytes, NK cells, other CD4+/CD8+ subsets) in the peripheral blood. All of these data provide new proposed mechanisms for these cytotoxic Temra CD8+ to home to the heart and recruit other immune cells to cause immune-mediated cardiac damage.

Our findings here also contribute to the broader understanding of ICI immune-mediated toxicities and provide a contrast of ICI myocarditis with irAE in other organ systems. The discovery of clonal expansion of T-cells after checkpoint blockade has been known for some time (36), and may even contribute to the therapeutic benefits of immune checkpoint blockade on tumor regression. However, distinguishing between the therapeutic expansion of clonal T-cell from a pathologic one that leads to autoimmune side effects will be necessary to prevent such toxicity with ICI treatment while retaining the benefits of immune checkpoint blockade. In a recent study using single-cell RNA-seq to examine patients with ICI-mediated colitis, Luoma et al also observed a CD8+ T-cell clonal expansion in the colonic tissues of these patients (37). Similar to the clonally expanded CD8+ cells in our patient population, the clonally expanded CD8+ cells in their patients were highly cytotoxic and activated (expressing high levels of GZMB and HLA-DRA), but expressed high levels of the mucosal associated chemokines CXCR3 and CXCR6, similar to some of our Group B patients. In contrast, the clonally expanded CD8+ cells in our Group C myocarditis patients expressed high levels of chemokines CCL4/CCL4L2/CCL5. The finding of organ-selective chemokine expression in the Temra CD8+ cells that bind to receptors enriched in heart tissues (CCR1/CCR5) of our myocarditis Group C patients may provide clues with regards to who may be at risk for developing cardiac-specific immune related events compared to the general population of patients experiencing irAE.

Due to the availability of early and late myocarditis blood samples, our study also provides new insight into the time-course of cellular and molecular changes that occur throughout the onset and resolution of ICI myocarditis. ICI myocarditis has a notoriously unpredictable time course and can occur with a median onset of 17-34 days after initiation of ICI therapy, although cases have been identified anywhere from 1 to 240 days from start of therapy (34,38-40). In our patient, we were able to collect blood at the time of diagnosis of the positive troponin I of 10 ng/ml (t=0) and track to day 65 after a full four-week course of glucocorticoids and resolution of clinical signs of myocarditis. The persistence of elevated clonal CD8+ Temra proportions (although attenuated) even at 65 days after diagnosis and treatment in this patient is intriguing and suggests additional T-cell mechanisms may be responsible for decreased pathogenicity over time, rather than steroid-mediated apoptosis of T-cells as previously described (41). The concept of T cell exhaustion is extremely important in immune checkpoint cancer biology, and the ability of ICI to re-invigorate exhausted T-cells to induce cytotoxicity against tumor tissue has been well-established (42). However, in the case of irAE, it would be desirable to reduce the cytotoxicity of T-cells that are damaging the normal tissue. Interestingly, our data suggests a phenotypic shift in the activated CD8+ Temra population, from an early cytotoxic and pro-inflammatory profile to a late exhaustion phenotype expressing known markers of T-cell exhaustion such as KLRG1 (43-45), CX3CR1 (46) and LAG3 (47) after glucocorticoid treatment. Indeed, T cell exhaustion has been described to play a central role in determining the outcome of many autoimmune diseases such as type I diabetes mellitus (48), and thus may also play a role in autoimmunity and response to anti-inflammatory treatment.

One of the challenges of diagnosing ICI myocarditis, and myocarditis at large, is the difficulty and risk of obtaining heart biopsy samples for accurate diagnosis. Our study provides several potential cellular and molecular biomarkers associated with ICI myocarditis in the peripheral blood. Peripheral blood has been shown to be a rich resource of immunologic information which may reflect immune changes at the tissue level, as demonstrated by prior studies from our group (49). Here, we use peripheral blood to glean insights into the state of inflammation in the heart in a rare patient population. However, it will be important to adjudicate and verify these biomarker targets in future studies using human heart tissue samples and/or animal studies. Other limitations for our data include the male and sex dominance of our data, which may be due to our small sample size. Unfortunately, because ICI myocarditis is a rare disease which occurs in approximately only ˜1% of patients on ICI (50), our myocarditis sample size is relatively small. However, this study contains the current largest single-cell immunologic dataset of confirmed ICI myocarditis cases to our knowledge, and thus is an important first step in this field with limited existing data. In larger epidemiologic studies, data have been conflicting regarding the association between ICI myocarditis and sex. Although 67-77% of myocarditis cases across several studies were reported in males (34,39,40), it is unclear whether this may reflect the disproportionate representation of men in ICI trials. Another study utilizing the FDA Adverse Event Reporting System demonstrated a higher incidence of ICI myocarditis in women (51), despite the classic association of autoimmune viral myocarditis with the male sex (52). Thus, these gender differences may be important to study in more detail going forward. Another limitation of our study is the relatively small sample size; to address this, we are continuing to recruit more patients, particularly women, for future single-cell transcriptomic analysis.

Our data give rise to important therapeutic targets for the treatment of ICI myocarditis and other ICI-related autoimmune side effects. The expression of T-cell exhaustion markers and upregulation of other immune checkpoints such as LAG3 suggest them as an intriguing therapeutic alternative to steroids. A precedent for this has been seen with abatacept, a CTLA4 agonist which has been used effectively to treat ICI myocarditis (53). Therapeutic targeting and upregulation of other immune checkpoints, however, may have implications for the long-term effectiveness of the ICI from a cancer perspective. Thus, the most intriguing drug targets from our dataset may be the chemokines (CCL4, CCL4L2, and CCL5), which are upregulated in our Temra CD8⁺ dataset and may provide approaches to target myocardial-specific pathways of inflammation while preserving the broader anti-tumor immune pathways activated by ICI. In addition to being expressed on the myocardium, CCR5 is a receptor expressed broadly on lymphocytes and functions as a port of entry for the HIV virus (54,55). Thus, the well-established CCR5 inhibitor, Maraviroc, already in clinical use for the treatment of patients with HIV with relatively few known side effects, may pose an example of a pharmacologic to explore in patients at risk for or with ICI myocarditis. Maraviroc and other CCR1/CCR5 inhibitors may provide attractive and specific therapeutic options in patients with ICI myocarditis whose current therapeutic options are limited to broad-spectrum glucocorticoids or T-cell suppressive therapies (tacrolimus, antithymocyte globulin, infliximab, etc.) (50). The further investigation of this chemokine pathway in addition to the other therapeutic targets identified in this study may lead to significant improvements in the cardiac safety of patients on ICI.

Lastly, our study contributes to the field of peripheral biomarkers of myocarditis at large. While myocarditis may be due to many different etiologies, there may be a common immune pathway leading to pathogenesis and myocardial damage (8), some of which may be exacerbated in the setting of ICI therapy (7,56). Effector CD8+ T-cells have long been thought to play a role in the pathogenesis of myocarditis (57,58), and these findings have also been reflected in our study in ICI myocarditis patients. Furthermore, we have identified a specialized type of effector CD8+ T-cells—the Temra CD8+ cells—to be particularly associated with ICI myocarditis. To our knowledge, our study is the largest study using single-cell techniques to immunophenotype myocarditis in the blood, utilizing this novel high-resolution technology to better understand peripheral immune changes in the setting of cardiac inflammation.

### REFERENCES

1. Moslehi J J, Salem J E, Sosman J A, Lebrun-Vignes B, Johnson D B. Increased reporting of fatal immune checkpoint inhibitor-associated myocarditis. Lancet [Internet]. 2018; 391 (10124): 933. Available from: http://dx.doi.org/10.1016/S0140-6736 (18) 30533-6
2. Mahmood S S, Fradley M G, Cohen J V., Nohria A, Reynolds K L, Heinzerling L M, et al. Myocarditis in Patients Treated With Immune Checkpoint Inhibitors. J Am Coll Cardiol. 2018;
3. Salem J, Manouchehri A, Moey M, Lebrun-vignes B, Bastarache L, Pariente A, et al. Articles Cardiovascular toxicities associated with immune checkpoint inhibitors: an observational, retrospective, pharmacovigilance study. Lancet Oncol [Internet]. 2018; 2045 (18): 1-11. Available from: http://dx.doi.org/10.1016/S1470-2045 (18) 30608-9
4. Caforio A L P. Myocarditis: endomyocardial biopsy and circulating anti-heart autoantibodies are key to diagnosis and personalized etiology-directed treatment. Available from: https://academic.oup.com/eurheartj/article/42/16/1618/6128538
5. Bracamonte-Baran W, Čiháková D. Cardiac autoimmunity: Myocarditis. In: Advances in Experimental Medicine and Biology. 2017.
6. Blanco-Domínguez R, Sánchez-Díaz R, de la Fuente H, Jiménez-Borreguero L J, Matesanz-Marín A, Relaño M, et al. A Novel Circulating MicroRNA for the Detection of Acute Myocarditis. N Engl J Med. 2021; 384 (21): 2014-27.
7. Grabie N, Lichtman A H, Padera R. T cell checkpoint regulators in the heart. Cardiovasc Res. 2019; 115 (5): 869-77.
8. Bockstahler M, Fischer A, Goetzke C C, Neumaier H L, Sauter M, Kespohl M, et al. Heart-Specific Immune Responses in an Animal Model of AutoimmuneRelated Myocarditis Mitigated by an Immunoproteasome Inhibitor and Genetic Ablation. Circulation. 2020; 1885-902.
9. Amioka N, Nakamura K, Kimura T, Ohta-Ogo K, Tanaka T, Toji T, et al. Pathological and clinical effects of interleukin-6 on human myocarditis. J Cardiol. 2021; (xxxx): 2-5.
10. Thavendiranathan P, Zhang L, Zafar A, Drobni Z D, Mahmood S S, Cabral M, et al. Myocardial T1 and T2 Mapping by Magnetic Resonance in Patients With Immune Checkpoint Inhibitor-Associated Myocarditis. J Am Coll Cardiol. 2021; 77 (12): 1503-16.
11. Zhang L, Reynolds K L, Lyon A R, Palaskas N, Neilan T G. The Evolving Immunotherapy Landscape and the Epidemiology, Diagnosis, and Management of Cardiotoxicity: JACC: CardioOncology Primer. JACC CardioOncology. 2021; 3 (1): 35-47.
12. Johnson D B, Balko J M, Compton M L, Chalkias S, Gorham J, Xu Y, et al. Fulminant Myocarditis with Combination Immune Checkpoint Blockade. N Engl J Med. 2016;
13. Touat M, Maisonobe T, Knauss S, Ben Hadj Salem O, Hervier B, Auré K, et al. Immune checkpoint inhibitor-related myositis and myocarditis in patients with cancer. Neurology [Internet]. 2018; 10.1212/WNL.0000000000006124. Available from: http://www.neurology.org/lookup/doi/10.1212/WNL.0000000000006124
14. Wang J, Okazaki I M, Yoshida T, Chikuma S, Kato Y, Nakaki F, et al. PD-1 deficiency results in the development of fatal myocarditis in MRL mice. Int Immunol. 2010;
15. Tarrio M L, Grabie N, Bu D-x., Sharpe A H, Lichtman A H. PD-1 Protects against Inflammation and Myocyte Damage in T Cell-Mediated Myocarditis. J Immunol. 2012;
16. Bendall S C, Simonds E F, Qiu P, Amir E D, Krutzik P O, Finck R, et al. Hematopoietic Continuum Linked references are available on JSTOR for this article: Single-Cell Mass Cytometry of Differential Immune and Drug Responses Across a Human Hematopoietic Continuum. Science (80-). 2011; 332 (6030): 687-96.
17. Newell E, Sigal N, Bendall S, Nolan G, Davis M M. Cytometry by Time-of-Flight Shows Combinatorial Cytokine Expression and Virus-Specific Cell Niches within a Continuum of CD8+ T Cell Phenotypes. Immunity. 2012; 36 (1): 142-52.
18. Szabo P A, Levitin H M, Miron M, Snyder M E, Senda T, Yuan J, et al. Single-cell transcriptomics of human T cells reveals tissue and activation signatures in health and disease. Nat Commun. 2019; 10 (1).
19. Gibellini L, De Biasi S, Porta C, Lo Tartaro D, Depenni R, Pellacani G, et al. Single-Cell Approaches to Profile the Response to Immune Checkpoint Inhibitors. Front Immunol. 2020; 11 (March): 1-18.
20. Leipold M, Maecker H. Phenotyping of Live Human PBMC using CyTOF™ Mass Cytometry. Bio-Protocol [Internet]. 2015; 5 (2): 1-6. Available from: http://www.bio-protocol.org/e1382
21. Russi A E, Brown M A. Mass Cytometry: Single Cells, Many Features. Cell. 2016; 165 (2): 255-69.
22. Gadalla R, Noamani B, MacLeod BL, Dickson R J, Guo M, Xu W, et al. Validation of CyTOF against flow cytometry for immunological studies and monitoring of human cancer clinical trials. Front Oncol. 2019; 9 (MAY): 1-13.
23. Chattopadhyay P K, Winters A F, Iii W E L, Laino A S, Woods D M. High-Parameter Single-Cell Analysis. Annu Rev Anal Chem. 2019; 12:411-30.
24. Han A, Glanville J, Hansmann L, Davis M M. Linking T-cell receptor sequence to functional phenotype at the single-cell level. Nat Biotechnol. 2014;
25. Tomic A, Tomic I, Dekker C L, Maecker H T, Davis M M. The FluPRINT dataset, a multidimensional analysis of the influenza vaccine imprint on the immune system. Sci Data. 2019 Dec. 1; 6 (1).
26. Wilk A J, Rustagi A, Zhao N Q, Roque J, Martinez-Colón GJ, McKechnie J L, et al. A single-cell atlas of the peripheral immune response in patients with severe COVID-19. Nat Med. 2020;
27. Butler A, Hoffman P, Smibert P, Papalexi E, Satijia R. Integrating single-cell transcriptomic data across different conditions, technologies, and species Andrew. Nat Biotechnol. 2018; 36 (5): 411-20.
28. Chen Z, Liu Y, Hu S, Zhang M, Shi B, Wang Y. Decreased Treg Cell and TCR Expansion Are Involved in Long-Lasting Graves' Disease. Front Endocrinol (Lausanne). 2021; 12 (April): 1-12.
29. Machado F S, Koyama N S, Carregaro V, Ferreira B R, Milanezi C M, Teixeira M M, et al. CCR5 plays a critical role in the development of myocarditis and host protection in mice infected with Trypanosoma cruzi. J Infect Dis. 2005; 191 (4): 627-36.
30. Damås JK, Eiken H G, Øie E, Bjerkeli V, Yndestad A, Ueland T, et al. Myocardial expression of CC- and CXC-chemokines and their receptors in human end-stage heart failure. Cardiovasc Res. 2000; 47 (4): 778-87.
31. Kelly K M, Tocchetti C G, Lyashkov A, Tarwater P M, Bedja D, Graham D R, et al. CCR5 inhibition prevents cardiac dysfunction in the SIV/macaque model of HIV. J Am Heart Assoc. 2014; 3 (2): 1-8.
32. Wensveen F M, Jelenčić V, Polić B. NKG2D: A master regulator of immune cell responsiveness. Front Immunol. 2018; 9 (MAR).
33. Efremova M, Vento-Tormo M, Teichmann S A, Vento-Tormo R. CellPhoneDB: inferring cell-cell communication from combined expression of multi-subunit ligand-receptor complexes. Nat Protoc [Internet]. Available from: https://doi.org/10.1038/s41596-020-0292-x
34. Johnson D B, Balko J M, Compton M L, Chalkias S, Gorham J, Xu Y, et al. Fulminant myocarditis with combination immune checkpoint blockade. N Engl J Med. 2016; 375 (18): 1749-55.
35. Takeshita M, Suzuki K, Kondo Y, Morita R, Okuzono Y, Koga K, et al. Multidimensional analysis identified rheumatoid arthritis-driving pathway in human T cell. Ann Rheum Dis. 2019; 78 (10): 1346-56.
36. Yost K, Satpathy A, Wells D, Qi Y, Wang C, Kageyama R, et al. Clonal replacement of tumor-specific T cells following PD-1 blockade. bioRxiv. 2019; 25 (8): 648899.
37. Luoma A M, Suo S, Williams H L, Sharova T, Sullivan K, Manos M, et al. Molecular Pathways of Colon Inflammation Induced by Cancer Immunotherapy. Cell. 2020; 182 (3): 655-671.e22.
38. Waliany S, Neal J W, Reddy S, Wakelee H, Shah S A, Srinivas S, et al. Myocarditis Surveillance With High-Sensitivity Troponin I During Cancer Treatment With Immune Checkpoint Inhibitors. JACC CardioOncology. 2021; 3 (1): 137-9.
39. Mahmood S S, Fradley M G, Cohen J V., Nohria A, Reynolds K L, Heinzerling L M, et al. Myocarditis in Patients Treated With Immune Checkpoint Inhibitors. J Am Coll Cardiol. 2018; 71 (16): 1755-64.
40. Salem J E, Manouchehri A, Moey M, Lebrun-Vignes B, Bastarache L, Pariente A, et al. Cardiovascular toxicities associated with immune checkpoint inhibitors: an observational, retrospective, pharmacovigilance study. Lancet Oncol. 2018; 19 (12): 1579-89.
41. Arbour K C, Mezquita L, Long N, Rizvi H, Auclin E, Ni A, et al. Impact of baseline steroids on efficacy of programmed cell death-1 and programmed death-ligand 1 blockade in patients with non-small-cell lung cancer. J Clin Oncol. 2018; 36 (28): 2872-8.
42. Sade-Feldman M, Yizhak K, Bjorgaard S L, Ray JP, de Boer C G, Jenkins R W, et al. Defining T Cell States Associated with Response to Checkpoint Immunotherapy in Melanoma. Cell. 2018 Nov. 1; 175 (4): 998-1013.e20.
43. Herndler-Brandstetter D, Ishigame H, Shinnakasu R, Plajer V, Stecher C, Zhao J, et al. KLRG1+Effector CD8+ T Cells Lose KLRG1, Differentiate into All Memory T Cell Lineages, and Convey Enhanced Protective Immunity. Immunity [Internet]. 2018; 48 (4): 716-729.e8. Available from: https://doi.org/10.1016/j.immuni.2018.03.015
44. Henson S M, Akbar A N. KLRG1-more than a marker for T cell senescence. Age (Omaha). 2009; 31 (4): 285-91.
45. Li L, Wan S, Tao K, Wang G, Zhao E. KLRG1 restricts memory T cell antitumor immunity. Oncotarget. 2016; 7 (38): 61670-8.
46. Sasaki M, Miyakoshi M, Sato Y, Nakanuma Y. Chemokine-chemokine receptor CCL2-CCR2 and CX3CL1-CX3CR1 axis may play a role in the aggravated inflammation in primary biliary cirrhosis. Dig Dis Sci. 2014; 59 (2): 358-64
47. Graydon C G, Mohideen S, Fowke K R. LAG3's Enigmatic Mechanism of Action. Front Immunol. 2021; 11 (January): 1-7.
48. Mckinney E F, Lee J C, Jayne D R W, Lyons P A, Smith K G C. T-cell exhaustion, co-stimulation and clinical outcome in autoimmunity and infection. Nature. 2015; 523 (7562): 612-6.
49. Zhao F, Sikora M J, Serratelli W S, Fernandes R A, Louis D M, Yao W, et al. Opposing T cell responses in experimental autoimmune encephalomyelitis.
50. Waliany S, Lee D, Witteles R M, Neal J W, Nguyen P, Davis M M, et al. Immune Checkpoint Inhibitor Cardiotoxicity: Understanding Basic Mechanisms and Clinical Characteristics and Finding a Cure. Annu Rev Pharmacol Toxicol. 2021; 61:113-34.
51. Zamami Y, Niimura T, Okada N, Koyama T, Fukushima K, Izawa-Ishizawa Y, et al. Factors Associated with Immune Checkpoint Inhibitor-Related Myocarditis. JAMA Oncology. 2019.
52. Kyto V, Sipila J, Rautava P. Gender differences in myocarditis: a nationwide study in Finland. Eur Heart J. 2013;
53. Salem J E, Allenbach Y, Kerneis M. Abatacept for severe immune checkpoint inhibitor-associated myocarditis. N Engl J Med. 2019; 380 (24): 2377-9.
54. Marques R E, Guabiraba R, Russo R C, Teixeira M M. Targeting CCL5 in inflammation. Expert Opin Ther Targets. 2013; 17 (12): 1439-60.
55. Crawford A, Angelosanto J M, Nadwodny K L, Blackburn S D, Wherry E J. A role for the chemokine RANTES in regulating CD8 T cell responses during chronic viral infection. PLOS Pathog. 2011; 7 (7).
56. Lichtman A H. The heart of the matter: Protection of the myocardium from T cells. Journal of Autoimmunity. 2013.
57. Henke A, Huber S, Stelzner A, Whitton J L. The role of CD8+T lymphocytes in coxsackievirus B3-induced myocarditis. J Virol. 1995; 69 (11): 6720-8.
58. Grabie N, Delfs M W, Westrich J R, Love V A, Stavrakis G, Ahmad F, et al. IL-12 is required for differentiation of pathogenic CD8+ T cell effectors that cause myocarditis. J Clin Invest. 2003; 111 (5): 671-80.
59. Ilicic T, Kim J K, Kolodziejczyk A A, Bagger F O, McCarthy D J, Marioni J C, et al. Classification of low quality cells from single-cell RNA-seq data. Genome Biol [Internet]. 2016; 17 (1): 1-15. Available from: http://dx.doi.org/10.1186/s13059-016-0888-1
60. Pijuan-sala B, Griffiths J A, Guibentif C, Hiscock T W, Jawaid W, Calero-nieto F J, et al. A single-cell molecular map of mouse gastrulation and early organogenesis. Nature. 2019; 566 (7745): 490-5.
61. Blondel V D, Guillaume J L, Lambiotte R, Lefebvre E. Fast unfolding of communities in large networks. J Stat Mech Theory Exp. 2008; 2008 (10).
62. Becht E, Mcinnes L, Healy J, Dutertre C-A, Kwok I W H, Ng L G, et al. Dimensionality reduction for visualizing single-cell data using UMAP. Nat Biotechnol. 2019; 37 (1).
63. Efremova M, Vento-Tormo M, Teichmann S A, Vento-Tormo R. CellPhoneDB: inferring cell-cell communication from combined expression of multi-subunit ligand-receptor complexes. Nat Protoc.

### List of Gene Abbreviations and Gene Information

GZMB stands for granzyme B, Gene ID: 3002 in the NIH gene library
PRF1 stands for perforin 1, Gene ID: 5551 in the NIH gene library
KLRK1 stands for killer cell lectin like receptor K1, Gene ID: 22914 in the NIH gene library
KLRB1 stands for killer cell lectin like receptor B1, Gene ID: 3820 in the NIH gene library
KLRF1 stands for killer cell lectin like receptor F1, Gene ID: 51348 in the NIH gene library
IL32 stands for interleukin 32, Gene ID: 9235 in the NIH gene library
CD45RA stands for protein tyrosine phosphatase receptor type C, Gene ID: 5788 in the NIH gene library
HLA-DRA stands for major histocompatibility complex, class II, DR alpha, Gene ID: 3122 in the NIH gene library
CCL4 stands for C-C motif chemokine ligand 4, Gene ID: 6351 in the NIH gene library
CCL4L2 stands for C-C motif chemokine ligand 4 like 2, Gene ID: 9560 in the NIH gene library
CCL5 stands for C-C motif chemokine ligand 5, Gene ID: 6352 in the NIH gene library
NKG2D is also known as KLRK1 which stands for killer cell lectin like receptor K1, Gene ID: 22914 in the NIH gene library
CXCR3 stands for C—X—C motif chemokine receptor 3, Gene ID: 2833 in the NIH gene library
CXCR6 stands for C—X—C motif chemokine receptor 6, Gene ID: 10663 in the NIH gene library
CTLA4 stands for cytotoxic T-lymphocyte associated protein 4, Gene ID: 1493 in the NIH gene library
CX3CR1 stands for C-X3-C motif chemokine receptor 1, Gene ID: 1524 in the NIH gene library
LAG3 stands for lymphocyte activating 3, Gene ID: 3902 in the NIH gene library
PD-1 stands for programmed cell death 1, Gene ID: 5133 in the NIH gene library
PD-L1 stands for CD274 molecule, Gene ID: 29126 in the NIH gene library
CTLA4 stands for cytotoxic T-lymphocyte associated protein 4, Gene ID: 1493 in the NIH gene library
CCR1 stands for C-C motif chemokine receptor 1, Gene ID: 1230 in the NIH gene library
CCR3 stands for C-X3-C motif chemokine receptor 3, Gene ID: 1232 in the NIH gene library
CCR5 stands for C-X3-C motif chemokine receptor 5, Gene ID: 1234 in the NIH gene library
TIGIT stands for T cell immunoreceptor with Ig and ITIM domains, Gene ID: 201633 in the NIH gene library
KLRG1 stands for killer cell lectin like receptor G1, Gene ID: 10219 in the NIH gene library
IL2RG stands for interleukin 2 receptor subunit gamma, Gene ID: 3561 in the NIH gene library
KLF2 stands for KLF transcription factor 2, Gene ID: 10365 in the NIH gene library
CD45 stands for protein tyrosine phosphatase receptor type C, Gene ID: 5788 in the NIH gene library
CD4 encodes the CD4 membrane glycoprotein of T lymphocytes, Gene ID: 920 in the NIH gene library
CD8 encodes the CD8 membrane glycoprotein of T lymphocytes, Gene ID: 925 in the NIH gene library

1. A method for reducing an immune-related adverse event (irAE) in an immune checkpoint inhibitor (ICI)-treated human subject, wherein the irAE includes ICI-induced myocarditis, the method comprising administering to the subject a composition comprising one or more of the following:
i. an inhibitor that antagonizes interaction of chemokine (C-C motif) ligand 5 (CCL5) with at least one of the following receptors: C-C chemokine receptor type 1 (CCR1), C-C chemokine receptor type 3 (CCR3) and/or C-C chemokine receptor type 5 (CCR5);
ii. an inhibitor that antagonizes action of the C-C chemokine receptor type 2 (CCR2);
iii. an antibody binding to and downregulating cytotoxic CD8+T effector cells that express one or more cytotoxicity markers and at least the following myocardial-tropic chemokines CCL3, CCL4, CCL4L2 and CCL5 (Temra CD8+ cells); and/or
iv. an antibody binding to and downregulating monocytes/macrophages that express one or more cytotoxicity markers and either C-C chemokine receptor type 2 (CCR2) or C-C chemokine receptor type 5 (CCR5).

2. The method of claim 1, wherein the method is further characterized by one or more of the following features:
the cytoxicity markers include one or more of the following cytoxicity markers: GZMB, PRF1, KLRK1, KLRB1, KLRF1, and/or IL32;
the Temra CD8⁺ cells express activation marker HLA-DRA;
the macrophages/monocytes expressing either CCR2 or CCR5;
the ICI-myocarditis was diagnosed on basis of elevated cardiac biomarker troponin I over the normal range for the general population, clinical syndrome, negative coronary work-up and/or an imaging diagnosis which included magnetic resonance imaging and/or positron emission tomography (PET); and/or
the reduction in the immune-related adverse event (irAE) is determined by measuring troponin levels prior to administering the composition and recorded as T1, and after administering the composition and recorded as T2, the reduction being achieved if T1 is greater than T2.

3. The method of claim 1, wherein the ICI is a monoclonal antibody or an antigen-binding fragment thereof, and wherein the antibody or the fragment selectively binds a regulatory protein present on a T cell, or a ligand for the protein, and thereby activates T-cell cytotoxicity against tumor cells.

4. The method of claim 3, wherein the regulatory protein is cytotoxic T-cell antigen-4 or programmed death 1 protein (PD-1) or wherein the ligand is programmed death 1 ligand (PDL-1).

5. The method of claim 1, wherein the ICI is one or more of the following: Ipilimumab (YERVOY), Nivolumab (OPDIVO), Pembrolizumab (KEYTRUDA), Atezolizumab (TECENTRIQ), Durvalumab (IMFINZI), Avelumab (BAVENCIO), and/or Cemiplimab-rwlc (LIBTAYO).

6. The method of claim 1, wherein the inhibitor of interaction of CCL5 includes one or more of the following:
an inhibitor of CCR1 selected from CP-481,715 (Pfizer), iMLN3897 (Millennium), BX471 (Berlex/Scherring AG) and AZD-4818 (Astra-Zeneca);
an inhibitor of CCR3 selected from SB297006, SB328437, and GW766994; and/or
an inhibitor of CCR5 selected from Maraviroc (monocarboxylic acid amide obtained by formal condensation of the carboxy group of 4,4-difluorocyclohexanecarboxylic acid and the primary amino group of (1S)-3-[(3-exo)-3-(3-isopropyl-5-methyl-4H-1,2,4-triazol-4-yl)-8-azabicyclo[3.2.1]oct-8-yl]-1-phenylpropylamine); leronlimab (PRO 140), cenicriviroc (CCR2/5 antagonist), and/or Met-CCL5 (CCL5 antagonist).

7. The method of claim 1, wherein the antibody binding to and downregulating the Temra CD8 cells is a monoclonal antibody specific to receptor CD45RA expressed on the Temra CD8⁺ cells.

8. The method of claim 7, wherein the antibody is selected from monoclonal antibody clone HI100 or 5H9 (BD Biosciences), or from clone T6D11 (Miltenyi).

9. The method of claim 1, wherein the composition is administered in a therapeutically effective concentration for a period of one to twenty one days, and wherein the therapeutically effective amount is sufficient to reduce the immune-related adverse event (irAE) as determined by measuring troponin levels prior to administering the composition and recorded as T1, and after administering the composition and recorded as T2, the reduction being achieved if T1 is greater than T2.

10. The method of claim 1, wherein prior to administering the composition, the subject is tested for levels of CCL5 in the subject's peripheral blood sample and/or for the levels of the Temra CD8⁺ cells in the subject's peripheral blood sample.

11. The method of claim 10, wherein the subject is tested for levels of CCL5 protein in the subject's peripheral blood sample.

12. The method of claim 1, wherein the method further comprises drawing a blood sample from the subject prior to administering the composition.

13. The method of claim 1, wherein the method further comprises:
drawing a blood sample from the subject prior to treating the subject with the ICI, after administering the ICI but before administering the composition and after administering the composition, and
measuring the CCL5 protein and/or mRNA levels in the samples;
measuring a number of the Temra CD8⁺ cells in the samples; and/or
measuring numbers of CCR2⁺ or CCR5⁺ monocytes/macrophages in the samples.

14. The method of claim 1, wherein the composition is administered at one or more of the following time periods: prior to treatment with the ICI, during the treatment with the ICI, and/or after the treatment with the ICI.

15. The method of claim 1, wherein the composition is administered to the subject in a therapeutically effective amount.

16. A method for testing a human subject undergoing treatment with an immune checkpoint inhibitor (ICI), the method comprising:
1) drawing a first blood sample from the human subject prior to administering the ICI;
2) drawing a second blood sample from the human subject after administering the ICI; and
3) measuring CCL5 protein and/or mRNA levels in the first sample and in the second sample; and/or measuring a number of specialized effector CD8⁺ T cells expressing one or more cytotoxicity markers and at least the following myocardial-tropic chemokines CCL4, CCL4L2 and CCL5 (Temra CD8+ cells) in the first sample and in the second sample and/or
4) measuring CCR2/CCR5 protein and/or mRNA levels in the first sample and in the second sample; and/or measuring a number of monocytes/macrophages expressing one or more cytotoxicity markers and at least the following chemokine receptors (CCR2 or CCR5) in the first sample and in the second sample.

17. The method of claim 16, wherein the level of CCL5 protein and/or mRNA in the first sample is L1, and wherein the level of CCL5 protein and/or mRNA in the second sample is L2, and wherein when L2 is greater than L1, the human subject is eligible for anti-irAE treatment.

18. The method of claim 16, wherein the method is further characterized by one or more of the following features:
the CCL5 protein, CCR2 protein, and/or CCR5 protein is detected by ELISA and/or CCL5 mRNA is detected by quantitative PCR; and/or
mononuclear cells are isolated from the blood samples, CD8⁺ T cells or monocytes/macrophages are isolated from the blood samples, and the CCL5 protein, CCR2 protein and/or CCR5 protein; and/or CCL5 mRNA, CCR2 mRNA and/or CCR5 mRNA is detected in the isolated population of CD8⁺ T cells and/or monocytes and/or macrophages.

19. The method of claim 16 , wherein the number of Temra CD8⁺ cells and/or monocytes/macrophages in the first sample is T1, and wherein the number of Temra CD8⁺ cells and/or monocytes/macrophages in the second sample is T2, and wherein when T2 is greater than T1, the subject is eligible for anti-irAE treatment.

20. (canceled)

21. (canceled)

22. (canceled)

23. (canceled)

24. A method of identifying a human subject eligible for ICI-myocarditis treatment, wherein the human subject is undergoing treatment with an immune checkpoint inhibitor (ICI), the method comprising analyzing CD8+ T cell population in a peripheral blood sample of the human subject for presence of Temra CD8+ cells expressing myocardial-tropic chemokines: CCL3, CCL4, CCL4L2 and CCL5 and also expressing CD45RA, and wherein when the Temra CD8 cells are present in the blood sample, the human subject is eligible for ICI-myocarditis treatment.

25. The method of claim 24, wherein the method further comprises one or more of the following:
the CD8+ T cell population is analyzed during a time period from day 15 to day 35 after initiation of the ICI therapy;
the analysis includes CyTOF (time of flight mass cytometry) and/or simultaneous single cell RNA-Seq and single cell TCR sequencing; and/or
the method further comprises collecting a blood sample before the ICI treatment begins, said sample being used as a control sample and wherein the human subject is eligible for ICI-myocarditis treatment when the presence of the Tempra CD8 cells is greater in the sample obtained during the ICI-treatment than in the human subject's control sample.

26. (canceled)

27. (canceled)

28. A method of treating ICI-induced myocarditis in a human subject, wherein the method comprises monitoring the human subject for a phenotypic shift in the activated Temra CD8+ population from an early cytotoxic and pro-inflammatory profile at the beginning of the ICI-induced myocarditis to a late exhaustion phenotype expressing markers of T-cell exhaustion, including KLRG, CX3CR1 and/or LAG3.
